(19) United States
(12) Patent Application Publication
(10) Pub. No.: US 2005/0036988 A1
(43) Pub. Date: Feb. 17, 2005

(54) Compositions and methods for preventing and treating liver cirrhosis

(76) Inventors: Ruian Xu, Hong Kong (HK); Xinyan Li, Nanjing (HK); Peter Chin Wan Fung, Pokfulam (HK)

Correspondence Address: Robert D. Katz, Esq.;Cooper & Dunham LLP, 1185 Avenue of the Americas, New York, NY, 10036, US

(21) Appl. No.: 10/839,936

(22) Filed: May 5, 2004

Related U.S. Application Data
(60) Provisional application No. 60/473,992, filed on May 28, 2003.

(51) Int. Cl. A61K 48/00

(52) U.S. Cl. 424/93.2; 514/44

(57) ABSTRACT

This invention provides a method for treating liver cirrhosis in a subject comprising administering to the subject a therapeutically effective amount of a rAAV/CAG-STAP vector, to treat liver cirrhosis in the subject. This invention further provides a method for preventing liver cirrhosis in a subject at risk for liver cirrhosis comprising administering to the subject a prophylactically effective amount of a rAAV/CAG-STAP vector thereby preventing liver cirrhosis in the subject. Finally, this invention provides related viral vectors and pharmaceutical compositions.

This application claims priority of U.S. Provisional Application No. 60/473,992, filed May 28, 2003, the contents of which are hereby incorporated by reference into this application.

Throughout this application, various publications may be referenced by author name and date in parentheses. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

### BACKGROUND OF THE INVENTION

Liver cirrhosis is a worldwide health problem. It is the irreversible end result of fibrous scarring, and is characterized by diffused disorganization of the normal liver structure of regenerative nodules and fibrotic tissue (Lee, 1997). It has become one of the leading causes of death by disease.

Hepatic cirrhosis is a disease resulting from hepatic chronic damage. Damage might be toxic (chronic ingestion of alcohol), infectious (viral hepatitis, mainly by hepatitis B and/or C virus), immunological, (primary biliary cirrhosis), by biliary obstruction, (secondary biliary cirrhosis) metabolic (Wilson's disease). All forms of cirrhosis have characteristics in common: synthesis and excessive deposition of proteins of extracellular matrix (ECM), mainly collagen I and to a lesser extent collagens IV and ll), and consequently the formation of nodules of hepatocytes, abnormal vascularization and portal hypertension. These physiopathological processes lead to an alteration in the blood supply and in consequence in the nutrition of hepatic cells. Regardless of the etiological agent and morphologic differences, all forms of cirrhosis have as a common end, hepatic failure causing the patient's death.

Incidence of cirrhosis is growing as a result of the widespread occurrence of chronic hepatitis and the obvious lack of an established therapy for hepatic fibrosis. It is estimated that 350 million people worldwide have chronic HBV infection (Xu et al., 2003b; Ueki et al., 1999). In Southeast Asia, Africa and China, more than 50% of the population is infected, and 8% to 15% have become chronically infected. Chronic HBV infection is the cause of up to 50% of cirrhosis cases in these regions (Xu et al., 2003b; Ueki et al., 1999). The resulting distortion of the liver architecture compromises the function of hepatocytes, causing systemic life-threatening complications.

Cirrhosis still remains untreatable by conventional therapy. Recent progress in vector development has heralded a possible treatment (Lee, 1997; Rudolph et al., 2000). However, the oncogenic potential of therapeutic genes, such as hepatic growth factor (HGF) (Ueki et al., 1999) and telomerase genes (Rudolf et al., 2000), might prevent their use in humans. The development of a new therapy for liver cirrhosis would be greatly facilitated by the availability of a suitable therapeutic gene for clinical trials.

A novel endogenous peroxidase gene, stellate cell activation-associated protein (STAP) was recently isolated from fibrotic liver and stellate cells. The potential of STAP in catabolizing hydrogen peroxide and lipid hydroperoxides has already been noted (Kawada et al., 2001). Since both have been reported to trigger HSC activation and can subsequently promote progression of liver fibrosis, the activation of hepatic stellate cells (HSC) is a key step for the development of liver cirrhosis. It is believed that oxidative stress plays an important role in the activation of transcription factors during activation of HSC. The experimental details that follow describe how STAP functions as an antifibrotic scavenger of peroxides during the progress of liver cirrhosis, and demonstrate the potential of STAP as a therapeutic gene for preventing or reversing exacerbated fibrosis, the most obvious hallmark of cirrhotic livers. The in vivo and primary culture approaches in this study are complementary for identifying regulatory mechanisms in stellate cell activation. The results provide a novel alternative therapeutic approach to liver cirrhosis.

Adeno-associated viruses (AAV) have been isolated from a number of species, including primates. They belong to the Parvoviridae family and have a single-stranded DNA genome. For its replicative life cycle, the AAV requires the presence of helper viruses such as adenovirus to replicate. In the absence of a helper virus, AAV integrates into the host genome and remains latent. When a latently infected cell encounters infection by a helper virus, the integrated AAV genome rescues itself and undergoes a productive lytic cycle. In recent years, several studies have demonstrated the efficacy of the rAAV gene delivery system for the treatment of multiple diseases in humans and animals.

AAV has several features that make it particularly useful for gene therapy. It is a defective, helper-dependent virus, and wildtype AAV is nonpathogenic in humans and other species. Vectors can be generated that are completely free of helper virus. Recombinant AAV vectors, with the entire coding sequence removed, retain only 145-base pair terminal repeats. These vectors, therefore, are devoid of all viral genes, minimizing any possibility of recombination and viral gene expression. Although AAV may induce immunological responses, these are relatively mild compared with the inflammation that accompanies early-generation adenoviral vectors. Major advantages of AAV vectors include stable integration, low immunogenicity, long-term expression, and ability to infect both dividing and nondividing cells; the major limitations include variations in infectivity of AAV among different cell types and the size of the recombinant genome that can be packaged. However, previous studies have demonstrated that AAV can be efficacious in hepatic gene therapy. In particular, Xu et al. have shown that AVV particles administered by hepatic portal vein injection can result in a high copy number in the liver and stable expression of the transgene (Xu et al., 2001).

To date no effective treatment of cirrhosis has been developed. The combination of an optimal promoter and gene delivery system with of an appropriate therapeutic gene is required to develop a highly efficient therapeutic and safe gene delivery system to treat liver fibrogenesis, to prevent chronic inflammation and to prevent the accumulation of cirrhotic tissue. The experimental details disclosed below provide a novel approach to prevention and treatment of liver cirrhosis.

### SUMMARY OF THE INVENTION

This invention provides a method for treating liver cirrhosis in a subject comprising administering to the subject a therapeutically effective amount of a rAAV/CAG-STAP vector, to treat liver cirrhosis in the subject.

This invention further provides a method for preventing or retarding the development of liver cirrhosis in a subject at risk for liver cirrhosis comprising administering to the subject a prophylactically effective amount of a rAAV/CAG-STAP vector to prevent or retard the development.

This invention further provides a method for treating liver cirrhosis in a subject afflicted with liver cirrhosis, comprising administering to the subject a therapeutically effective amount of a gene encoding the stellate cell activation-associated protein (STAP), to treat cirrhosis in the subject.

This invention further provides a method for preventing or retarding the development of liver cirrhosis in a subject at risk for liver cirrhosis, comprising administering to the subject a prophylactically effective amount of a gene encoding the stellate cell activation-associated protein (STAP), to prevent of retard liver cirrhosis in the subject.

This invention further provides a first viral vector comprising the rAAV/CAG-rat STAP vector (CCTCC Patent Deposit Designation V200306).

This invention further provides a kit comprising the first instant viral vector and instructions for use.

This invention further provides a second viral vector comprising the rAAV/CAG-human STAP vector (CCTCC Patent Deposit Designation V200305).

This invention further provides a kit comprising the second instant viral vector and instructions for use.

This invention further provides a first pharmaceutical composition comprising the first instant viral vector and a pharmaceutically acceptable carrier.

This invention further provides a second pharmaceutical composition comprising the second instant viral vector and a pharmaceutically acceptable carrier.

Finally, this invention provides a method for treating liver cirrhosis in a subject comprising administering to the subject a therapeutically effective amount of a viral vector including an antioxidant gene, to treat liver cirrhosis in the subject.

### BRIEF DESCRIPTION OF THE FIGURES

FIGS. 1A-1H

FIGS. 1A and 1B. rAAV/CAG-STAP vector diagram: (A) rAAV/CAG-rat STAP (CCTCC Patent Deposit Designation V200306) and (B) rAAV/CAG-human STAP (CCTCC Patent Deposit Designation V200305). FIGS. 1C and 1D. In situ hybridization to the liver sections by DIG immunological detection kit: (1C) non-transduced rats and (1D) rats transduced with rAAV/CAG-rat STAP for one month. FIGS. 1E-1H. Immunohistochemistry staining of liver sections from (1E) rats transduced with rAAV/CAG-EGFP, (1F) non-transduced rats (i.e., rats treated with PBS), (1G) rats transduced with rAAV/CAG-rat STAP, and (1H) rats transduced with rAAV/CAG-human STAP for 10 weeks.

FIGS. 2A-2F

Livers of (FIGS. A and B) non-transduced rats and no CCl₄ treatment, (Figures C and D) non-transduced and CCl₄ treated (8 weeks) rats (Figures E and F) rats transduced with rAAV/CAG-rat STAP for two weeks prior to treatment with CCl₄ for 8 weeks.

FIGS. 3A-3J

FIGS. 3A-3D. Masson's trichrome-stained liver sections taken (A) non-transduced and no CCl₄ treatment rats, (B) rats transduced with 3×10¹¹ rAAV/EGFP particles/animal and then treated with CCl₄ for 8 consecutive weeks, (C) rats treated with CCl₄ for 8 consecutive weeks, and (D) rats transduced with 3×10¹¹ rAAV/CAG-rat STAP particles/animal for 2 weeks prior to treatment with CCl₄ for 8 weeks. FIG. 3E. Analysis of fibrosis using an imaging analysis techniques, calculating the ratio of connective tissue to the whole area of the liver from the non-transduced and no CCl₄ treatment rats, rats transduced with 3×10¹¹ rAAV/CAG-rat STAP particles/animal for 2 weeks prior to treatment with CCl₄ for 8 weeks, rats transduced with 3×10¹¹ rAAV/EGFP particles/animal and then treated with CCl₄ for 8 weeks, and non-transduced rats treated with CCl₄ for 8 weeks. Values are presented as mean±standard deviation. FIG. 3F. RT-PCR analysis of PC-1 mRNA levels in total RNA samples extracted from the liver of different experimental animals (lanes 1 and 2: non-transduced and no CCl₄ treatment rats, lanes 3 and 4: non-transduced and CCl₄ treated (8 weeks) rats, lanes 5 and 6: rats transduced with rAAV/CAG-human STAP particles for two weeks prior to treatment with CCl₄ for 8 weeks, lanes 7 and 8: rats transduced with rAAV/CAG-rat STAP particles for two weeks prior to treatment with CCl₄ for 8 weeks). FIG. 3G. RT-PCR analysis of PC-3 mRNA levels in total RNA samples extracted from the liver of different experimental animals (lane 1: rat transduced with rAAV/CAG-human STAP particles for two weeks prior to treatment with CCl₄ for 8 weeks, lane 2: rat transduced with rAAV/CAG-rat STAP particles for two weeks prior to treatment with CCl₄ for 8 weeks, lane 3: non-transduced and CCl₄ treated (8 weeks) rat, and lane 4: non-transduced and no CCl₄ treatment rat). FIG. 3H. RT-PCR analysis of T11 mRNA levels in total RNA samples extracted from the liver of different experimental animals (lane 1: rat transduced with rAAV/CAG-human STAP particles for two weeks prior to treatment with CCl₄ for 8 weeks, lane 2: rat transduced with rAAV/CAG-rat STAP particles for two weeks prior to treatment with CCl₄ for 8 weeks, lane 3: non-transduced and CCl₄ treated (8 weeks) rat, and lane 4: non-transduced and no CCl₄ treatment rat). FIGS. 3I and 3J. TUNEL staining of sections taken from the livers of (I) rats transduced with rAAV/CAG-rat STAP particles and then treated with CCl₄ and (J) non-transduced rats treated with CCl₄.

FIGS. 4A-4H

FIGS. 4A-4E. Liver sections taken from rats treated with CCl₄ for 8 weeks followed by (4A, 4C and 4E) treatment with PBS or by (4B, 4D and 4F) transduction with rAAV/CAG-rat STAP particles. Immuno-staining with TGF-β1 antibody (FIGS. 4A and 4B), α-SMA antibody (FIGS. 4C and 4D), and PNCA antibody (FIGS. 4E and 4F). Western blot analysis of liver extracts with α-SMA antibody (FIG. 4G; lane 1: non-transduced and no CCl₄ treatment rat, lane 2: rat transduced with rAAV/CAG-rat STAP particles for two weeks prior to treatment with CCl₄ for 8 weeks, lane 3: rat transduced with rAAV/CAG-human STAP particles for two weeks prior to treatment with CCl₄ for 8 weeks, lane 4: rat transduced with rAAV/CAG-EGFP particles for two weeks prior to treatment with CCl₄ for 8 weeks, and lane 5 rat treated with PBS for two weeks prior to treatment with CCl₄ for 8 weeks), TGF-β1 antibody (FIG. 4H; lane 1: non-transduced and no CCl₄ treatment rat, lane 2: rat transduced with rAAV/CAG-rat STAP particles for two weeks prior to treatment with CCl₄ for 8 weeks, lane 3: rat transduced with rAAV/CAG-EGFP particles for two weeks prior to treatment with CCl₄ for 8 weeks, and lane 4 rat transduced with rAAV-CAG-EGFP for two weeks prior to treatment with CCl₄ for 8 weeks).

FIGS. 5A-5G

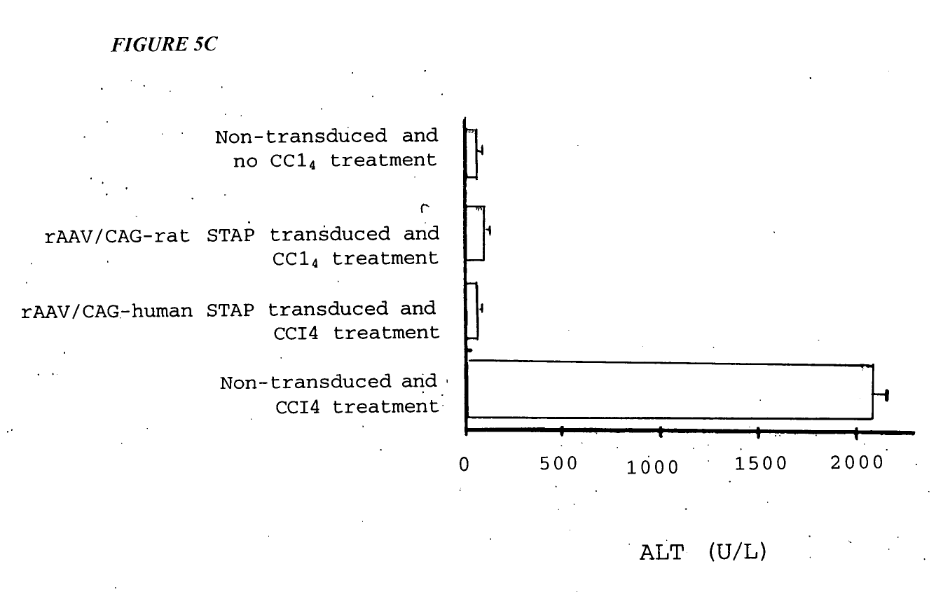

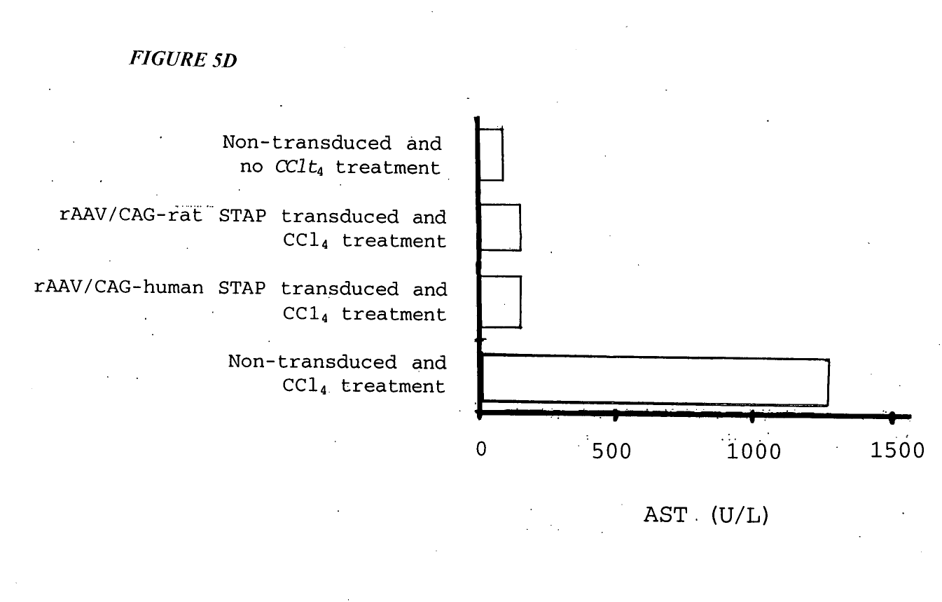

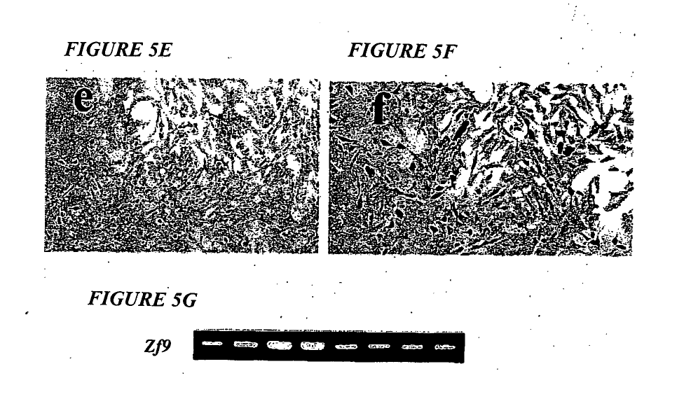

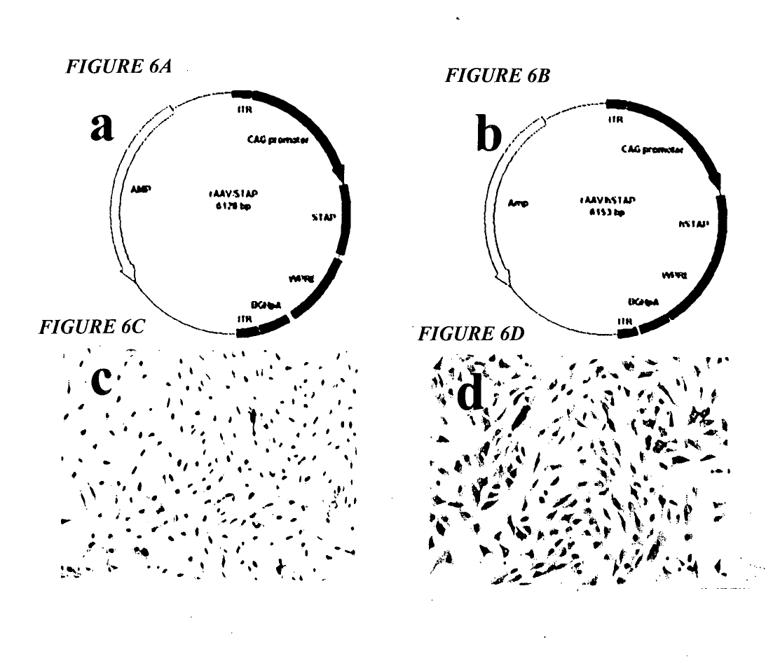

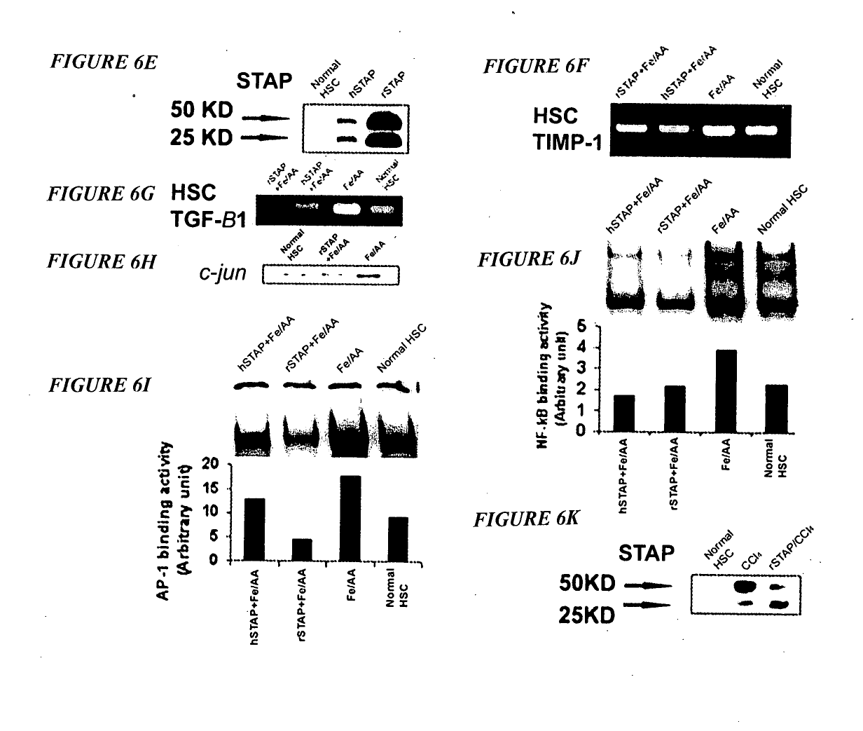

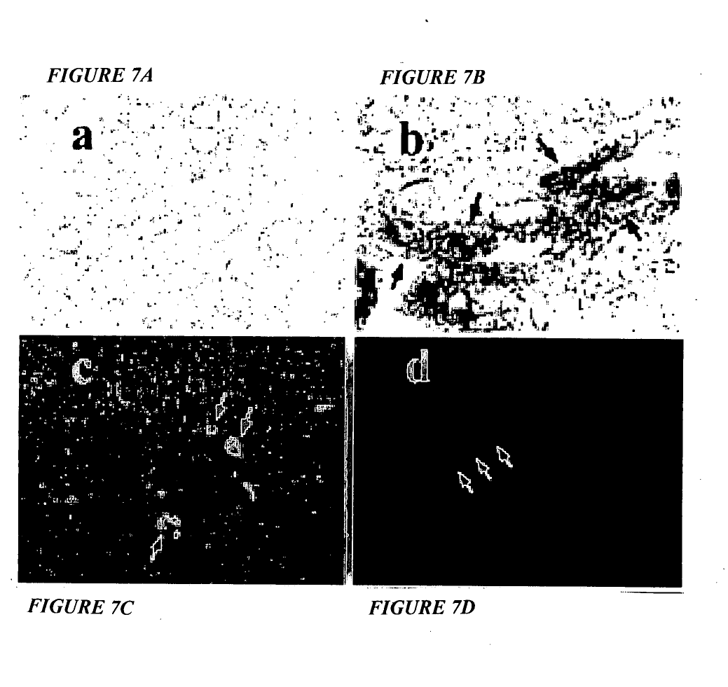

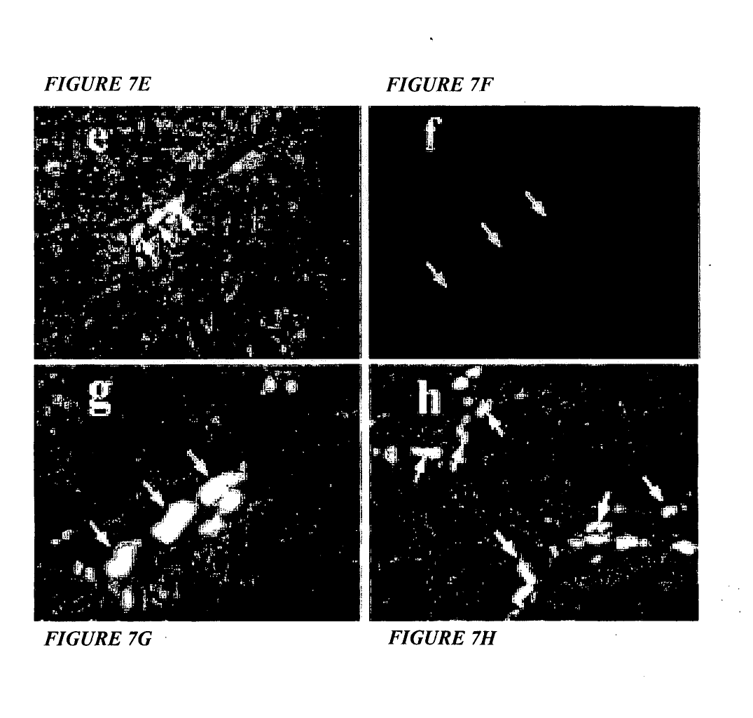

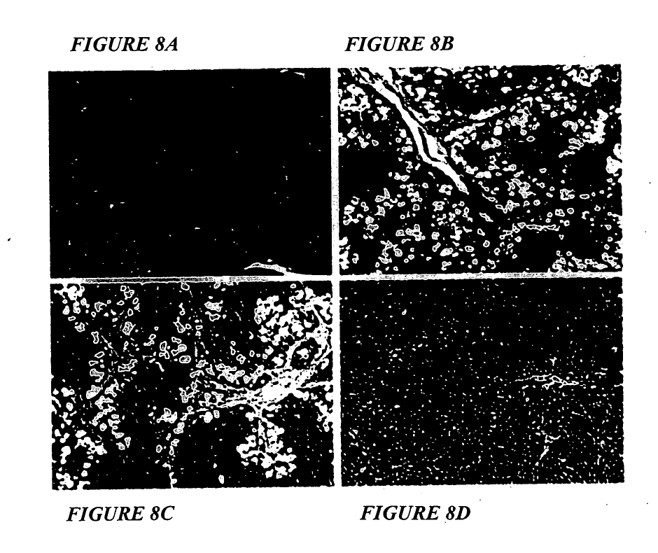

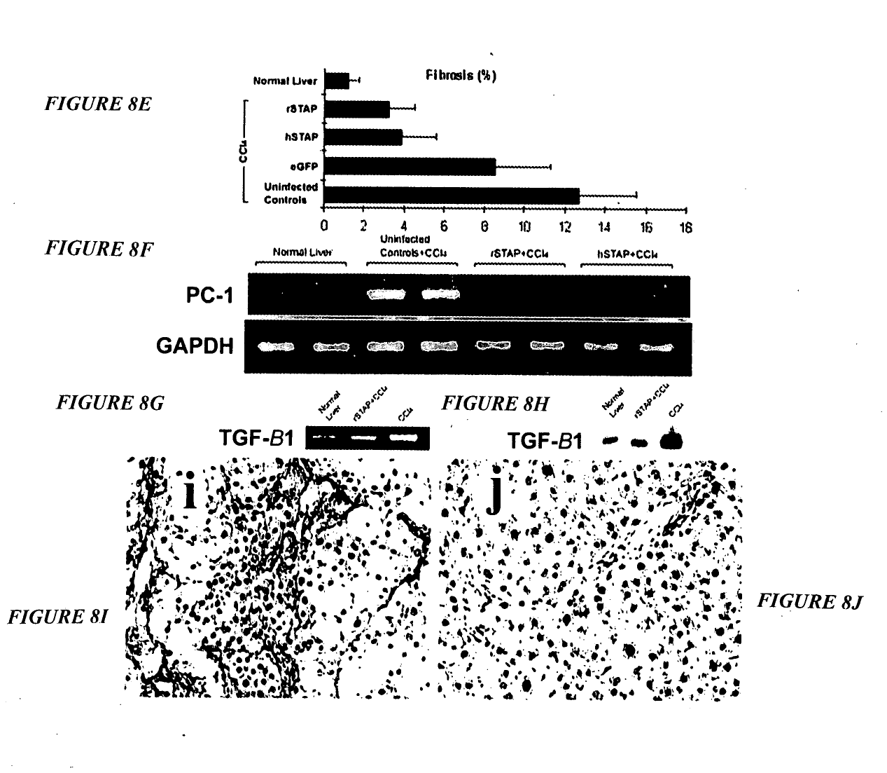

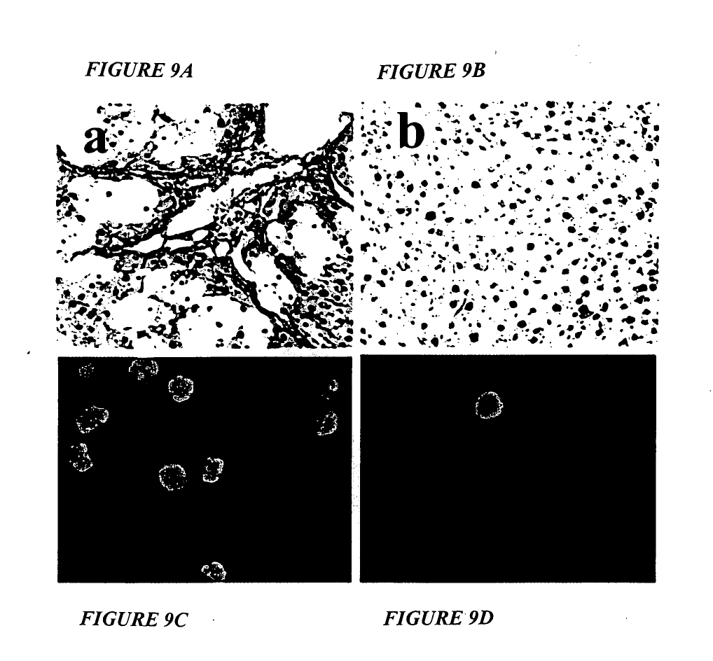

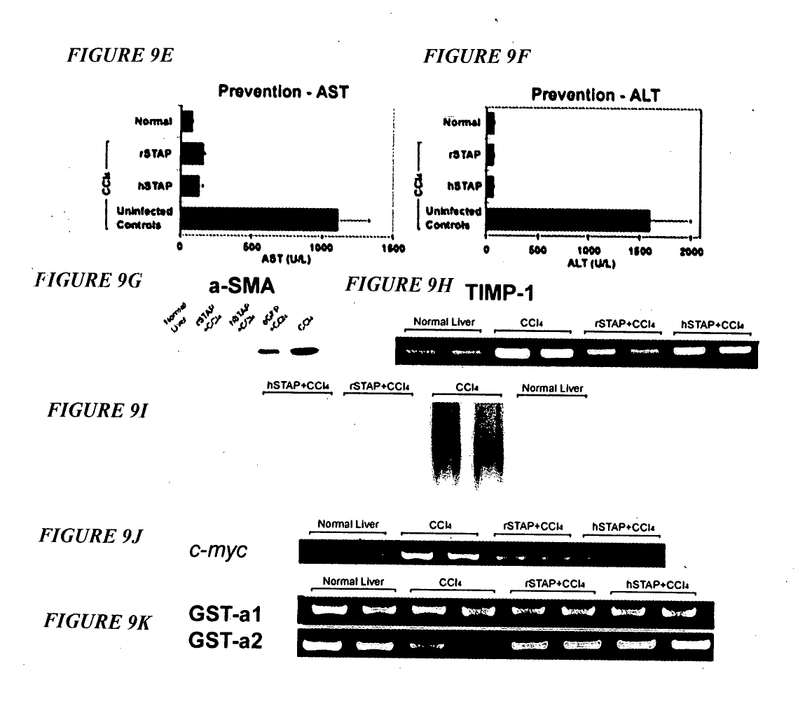

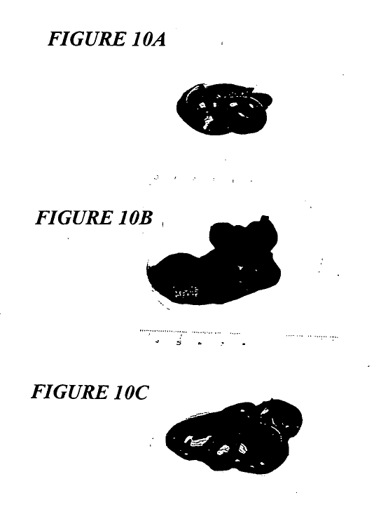

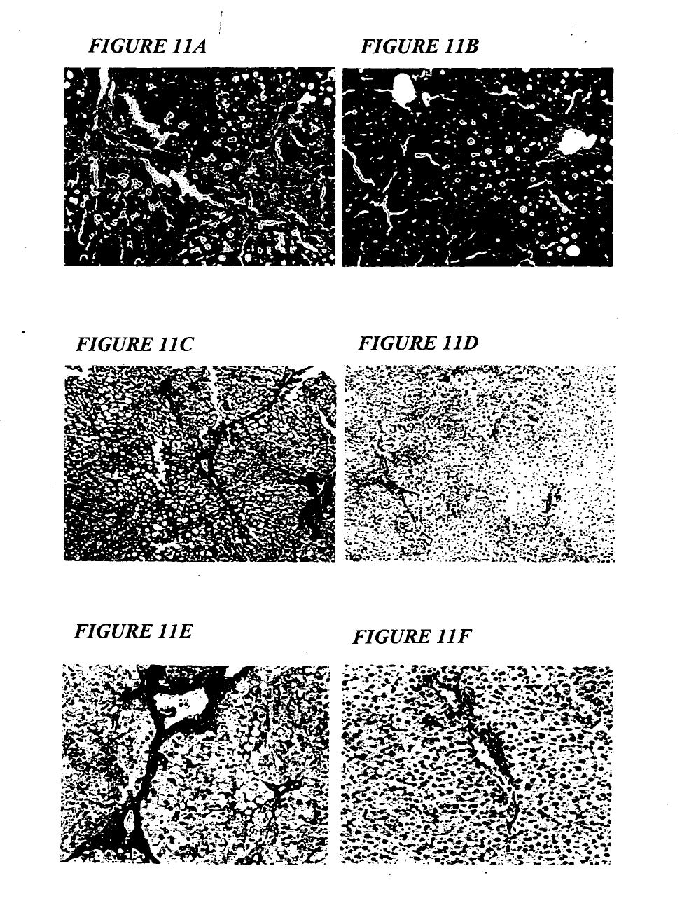

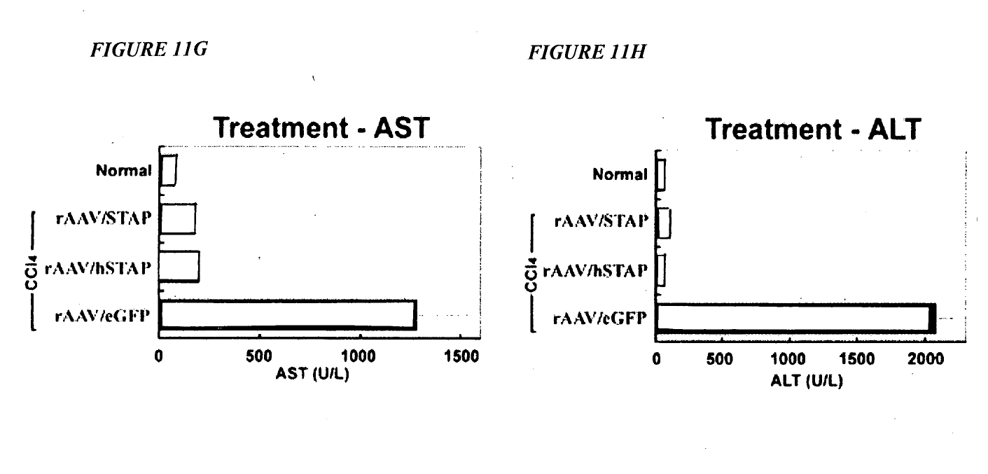

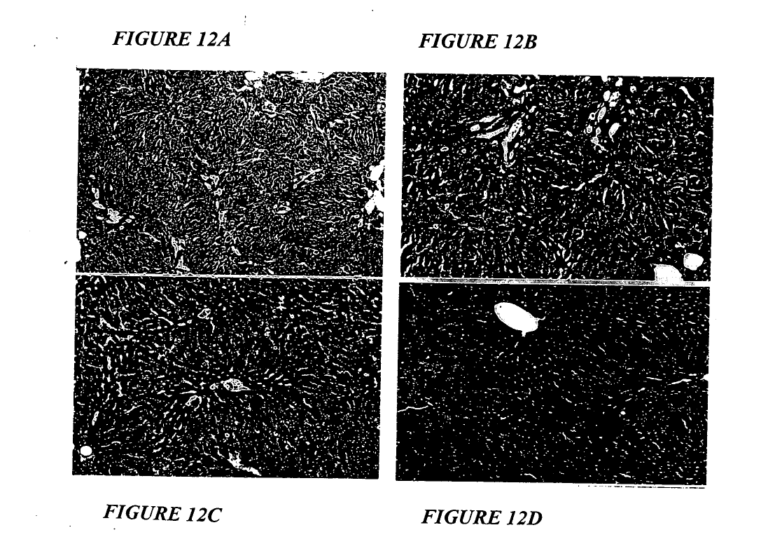

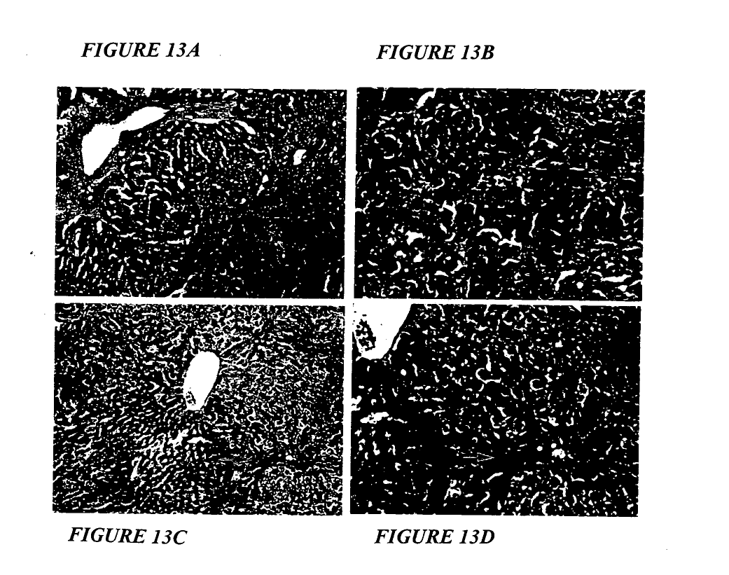

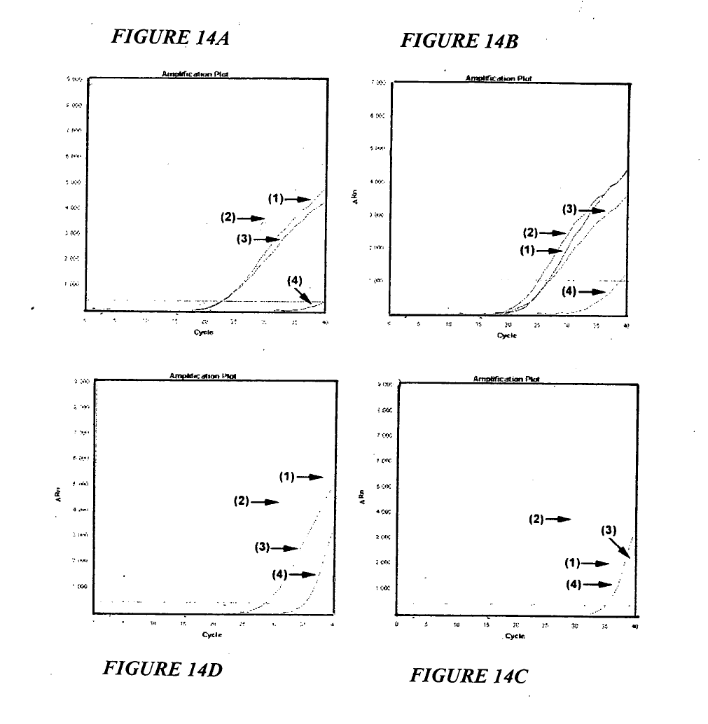

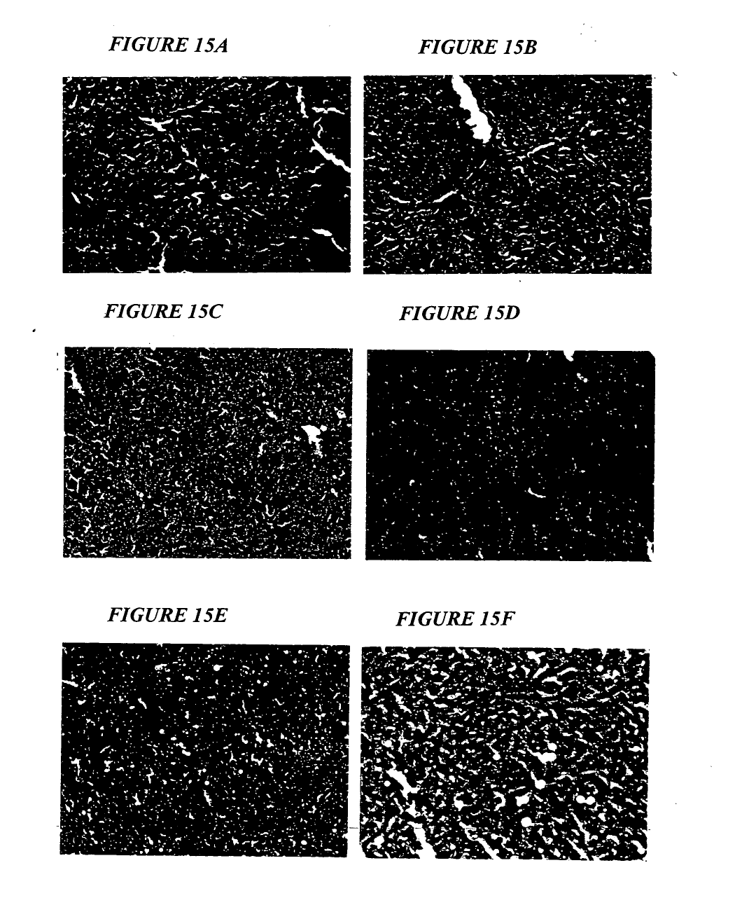

Levels of ALT (FIGS. 5A and 5C) and AST (FIGS. 5B and 5D) in non-transduced and no CCl₄ treatment rats, rAAV/CAG-rat STAP transduced rats treated with CCl₄, rAAV/CAG-human STAP transduced rats treated with CCl₄, rAAV/EGFP transduced rats treated with CCl₄ and non-transduced rats treated with CCl₄. FIGS. 5E and 5F. Immunostaining of primary stellate cells transduced with rAAV/EGFP (FIG. 5E) or transduced with rAAV/CAG-rat STAP (FIG. 5F) particles for two days. Cells were culture at 37° C. for three days prior to transduction. STAP positive cells (dark) were observed only in rAAV/CAG-rat STAP transduced primary stellate cells. FIG. 5G. RT-PCR analysis of Zf9 mRNA levels in total RNA extracted from the livers of different experimental animals lanes 1 and 2: non-transduced and no CCl₄ treatment rat, lane 3 and 4: rats treated with PBS for two weeks prior to treatment with CCl₄ for 8 weeks, lanes 5 and 6: rats transduced with rAAV/CAG-human STAP particles for two weeks prior to treatment with CCl₄ for 8 weeks, Lanes 7 and 8 rats transduced with rAAV/CAG-rat STAP particles for two weeks prior to treatment with CCl₄ for 8 weeks.

FIGS. 6A-6K

rAAV-2 mediated infection of primary HSC in vitro—rAAV/CAG-STAP vectors encoding rat (a) and human (b) STAP. STAP immunostaining of cultured primary HSC transduced with rAAV/eGFP (c) and rAAV/rSTAP for two days (d); HSC were cultured for three days prior to rAAV transduction. STAP positive cells (brown, ˜90%) were present in the rAAV/STAP infected HSC only. (e) Immunoblotting for STAP in normal and the rAAV/hSTAP or rAAV/rSTAP (MOI: 5×10⁴) for two days. (f) RT-PCR mediated quantification of TIMP-1, and TGF-β1 (g), in the Fe/AA treated control and STAP transduced HSC cells. (h) Immunoblotting for c-jun indicates STAP mediated inhibition of Fe/AA induced increase in c-Jun protein levels. Electrophoretic gel mobility shift analysis of AP-1 (i) or NF-kB (j) binding activity in the normal HSC and Fe/AA treated HSC, either without or with prior infection with rAAV vectors encoding either rat or human STAP. (k) Immunoblotting for STAP in rat liver tissue lysates indicates the absence of a detectable level of monomeric STAP in the normal, but increased levels of both the monomeric and the dimeric forms of STAP in the CCl₄ treated rat liver samples either in the absence or following the prior infection with the rAAV/rSTAP vector.

FIGS. 7A-7H

In vivo transduction of HSC by rAAV-2 vectors—DIG-non-radioactive in situ hybridization histochemistry (ISHH) for STAP RNA transcripts in liver sections by alkaline phosphatase NBT-BCIP detection kit (BM): normal rats (a) and rats one month after infection with rAAV/rSTAP (rSTAP) (b). Arrows indicate the positively stained cells. Double immunofluorescent labeling using antibodies to STAP (green in c, e-h) and to desmin (Sigma, red in d-h) or both (yellow in c, e, g and h) on the liver sections of normal rats (c) one month after treatment with rAAV/rSTAP vectors (d-g from same sample; d and e 400×, f and g, 800×) and CCl₄-control rats (h). Arrows indicate desmin positive cells. The primary antibodies used were mouse anti-desmin antibody (1:100) and rabbit anti-STAP antibody (1:200). The secondary antibodies were Cy5 conjugated donkey anti-mouse IgG (1:100) and FITC conjugated goat anti-rabbit IgG (1:100).

FIGS. 8A-8J

STAP gene expression prevents chronic CCl₄ induced liver cirrhosis—Masson's trichrome-stained liver sections from the normal (a), CCl₄-rAAV/EGFP (eGFP) (b), CCl₄-Control (CCl₄) (c) and CCl₄-rAAV/rSTAP (rSTAP) (d) rats. Analysis of fibrosis index (e) using an imaging analysis technique⁶, was used to calculate the ratio of the area of connective tissue to the total area of liver section in the normal control and in CCl₄ treated animals that were two weeks earlier infected with the rAAV-2 vectors encoding rSTAP, hSTAP, or eGFP. Values are presented as mean±standard deviation. (f) RT-PCR analysis of total RNA extracted from the liver with PC-1 primers in duplicate samples. Analysis of TGF-β1 expression by RT-PCR (g) and western-blotting (h) of liver samples isolated from the normal controls and the CCl₄ treated animals with or without prior rAAV/rSTAP infection, as indicated. Liver sections immunostained with TGF-β1 antibody (i: CCl₄-control; j: CCl₄-rAAV/rSTAP).

FIGS. 9A-9K

Inhibition of hepatic cell apoptosis and suppression of serological markers of liver cirrhosis by ectopic expression of STAP—Liver sections immunostained with A-SMA antibodies (a: CCl₄-control; b: CCl₄-rAAV/rSTAP). TUNEL staining of liver sections taken from the CCl₄ treated animals either without (c) or with prior infection with rAAV/rSTAP (d). Serum AST (e) and ALT (f) levels in the normal controls and in the CCl₄ treated animals either without or with prior infection with rAAV/rSTAP. (g) Western immunoblotting of liver extracts from different animals with α-SMA antibody. (i) Analysis of AP-1 binding activity by EMSA. The experimental conditions were identical to that used for the study of HSC (see FIG. 6). RT-PCR assessment of the transcript levels in the liver extracts for TIMP-1 (h), c-myc (j) and GST-α1 or GST-α2 (k), respectively, in duplicate samples.

FIGS. 10A-10C.

Inhibition of damage induced liver enlargement and fibrotic morphology by transgenic expression of STAP—Representative photographs of livers isolated from normal rats (a) and CCl₄-treated animals either without (b) and or with prior infection, 2 weeks earlier, with rAAV/rSTAP (c).

FIGS. 11A-11H

STAP gene expression attenuates exacerbated hepatic fibrosis—Liver sections of CCl₄-rAAV/eGFP (a, c &e), CCl₄-rAAV/rSTAP (b & f) and CCl₄-rAAV/hSTAP (d) rats, Masson's trichrome-stained (a & b) immunostained with α-SMA (c: CCl₄-rAAV/eGFP d: CCl₄-rAAV/hSTAP) and TGF-β1 antibodies (e: CCl₄-rAAV/eGFP; f: CCl₄-rAAV/rSTAP). Serum AST (g) and ALT (h) levels in the normal controls and in the 12-week-CCl₄ treated rats four weeks after infected with rAAV/rSTAP, rAAV/hSTAP or rAAV/EGFP respectively.

FIGS. 12A-12D

STAP administration attenuates ongoing liver fibrosis induced by common bile duct obstruction. Liver sections of BDL-eGFP (A); BDL-PBS (B), sham (C) and BDL-STAP (D) rats, Masson's trichrome-stained. Male SD rats were injected with 5×10¹¹ rAAV/rSTAP and rAAV/EGFP particles/animal respectively for three days prior to bile duct ligation. Animals were sacrificed 28 days after bile duct ligation.

FIGS. 13A-13D

Overexpression of STAP in HSC to prevent progressive liver damage by bile duct ligation Male SD rats were first exposed to BDL (12 days) and then injected via the portal vein with either PBS (B) or rAAV/eGFP (A) or rAAV/STAP (C, D) vectors. Liver sections were prepared 12 days (BDL animals) after the rAVV infections. Masson's trichrome-stained sections demonstrate prevention of BDL induced liver damage.

FIGS. 14A-14D

Real-time RT-PCR analysis of TGFβ-1 and PC-1 mRNA levels in HSC isolated at the time of sacrifice shows the activated phenotype of the HSC in the BDL animals and the quiescent phenotype of the HSC in the rAAV/rSTAP infected animals (1: sham operated; 2: BDL-rAAV/EGFP; 3: BDL-rAAV/rSTAP; 4: no template control).

FIGS. 15A-15E

Long effect of STAP in transgenic rats. Liver sections of CCl₄-rAAV/EGFP (A & B), normal (C & D) and CCl₄-rAAV/rSTAP (E & F) and rats, Masson's trichrome-stained. The 8-week-CCl₄ treated rats were injected with rAAV/rSTAP, rAAV/eGFP respectively and animals were continuously subjected to CCl4 induction for consecutive 4 weeks, and these animals and normal rats were all kept under identical conditions for another 40 week prior to sacrifice.

### DETAILED DESCRIPTION OF THE PREFERRED EMBODIMENTS

This invention provides a method for treating liver cirrhosis in a subject comprising administering to the subject a therapeutically effective amount of a rAAV/CAG-STAP vector, to treat liver cirrhosis in the subject.

In one specific embodiment, the rAAV/CAG-STAP vector transduces hepatic stellate cells.

In one specific embodiment the transduction of hepatic stellate cells results in the suppression of α-SMA, collagen, and/or TGF-β expression.

In one specific embodiment, the rAAV/CAG-STAP vector comprises the rat STAP sequence. In another specific embodiment, the rAAV/CAG-STAP vector comprises rAAV/CAG-rat STAP vector (CCTCC Patent Deposit Designation V200306). In another specific embodiment the rAAV/CAG-STAP vector comprises the human STAP sequence. In another specific embodiment, the rAAV/CAG-STAP vector comprises rAAV/CAG-human STAP vector (CCTCC Patent Deposit Designation V200305).

In one specific embodiment, the subject is a human. In another specific embodiment, the subject is a mammal. In the preferred embodiment the subject is a human.

In one specific embodiment the transduction of hepatic stellate cells inhibits fibrogenesis, hepatocyte apoptosis, or both.

In another specific embodiment transduction of hepatocytes with STAP reduces ALT and AST levels.

This invention further provides a method for preventing or retarding the development of liver cirrhosis in a subject at risk for liver cirrhosis comprising administering to the subject a prophylactically effective amount of a rAAV/CAG-STAP vector to prevent or retard the development of liver cirrhosis in the subject.

In one specific embodiment the rAAV/CAG-STAP vector transduces hepatic stellate cells. In another specific embodiment the transduction of hepatic stellate cells results in the suppression of α-SMA, collagen, and/or TGF-β expression.

In one specific embodiment the rAAV/CAG-STAP vector comprises the rat STAP sequence. In another specific embodiment the rAAV/CAG-STAP vector comprises rAAV/CAG-rat STAP vector (CCTCC Patent Deposit Designation V200306). In another specific embodiment the rAAV/CAG-STAP vector comprises the human STAP sequence. In another specific embodiment the rAAV/CAG-STAP vector comprises rAAV/CAG-human STAP vector (CCTCC Patent Deposit Designation V200305).

In one specific embodiment the subject is a mammal. In the preferred embodiment the mammal is human.

In one specific embodiment the transduction of hepatic stellate cells inhibits fibrogenesis, hepatocyte apoptosis, or both. In another specific embodiment transduction of hepatocytes with STAP reduces ALT and AST levels.

This invention further provides a method for treating liver cirrhosis in a subject afflicted with liver cirrhosis, comprising administering to the subject a therapeutically effective amount of a gene encoding the stellate cell activation-associated protein (STAP), to treat cirrhosis in the subject.

This invention further provides a method for preventing or retarding the development of liver cirrhosis in a subject at risk for liver cirrhosis, comprising administering to the subject a prophylactically effective amount of a gene encoding the stellate cell activation-associated protein (STAP), to prevent or retard the development of liver cirrhosis in the subject.

This invention further provides a first viral vector comprising the rAAV/CAG-rat STAP vector (CCTCC Patent Deposit Designation V200306).

This invention further provides a kit comprising the first instant viral vector and instructions for use.

This invention further provides a second viral vector comprising the rAAV/CAG-human STAP vector (CCTCC Patent Deposit Designation V200305).

This invention further provides a kit comprising the second instant viral vector and instructions for use.

This invention further provides a first pharmaceutical composition comprising the first instant viral vector and a pharmaceutically acceptable carrier.

This invention further provides a second pharmaceutical composition comprising the second instant viral vector and a pharmaceutically acceptable carrier.

Finally, this invention provides a method for treating liver cirrhosis in a subject comprising administering to the subject a therapeutically effective amount of a viral vector including an antioxidant gene, thereby treating liver cirrhosis in the subject.

In one embodiment, the viral vector transduces hepatic stellate cells. In another embodiment, the antioxidant gene is catalase. In another embodiment, the antioxidant gene is STAP.

Set forth below are certain additional definitions and examples which are intended to aid in an understanding of the instant invention.

“Administering” an agent can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

“Amino acid sequence” as used herein refers to an oligopeptide, peptide, polypeptide, or protein sequence, and fragments or portions thereof, and to naturally occurring or synthetic molecules. As used herein, the following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine; P=pro=proline; S=ser=serine; T=thr=threonine; W=trp=tryptophan; Y=tyr=tyrosine; V=val=valine; B=asx=asparagine or aspartic acid; Z=glx=glutamine or glutamic acid.

A “construct” is used to mean recombinant nucleic acid which may be a recombinant DNA or RNA molecule, that has been generated for the purpose of the expression of a specific nucleotide sequence(s), or is to be used in the construction of other recombinant nucleic acids. In general, “construct” is used herein to refer to an isolated, recombinant DNA or RNA molecule.

As used herein, the term “exogenous gene” refers to a gene that is not naturally present in a host organism or cell, or is artificially introduced into a host organism or cell.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the term “genome” refers to the genetic material (e.g., chromosomes) of an organism.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “multiplicity of infection” or “MOI” refers to the ratio of integrating vectors: host cells used during transfection or transduction of host cells. For example, if 1,000,000 vectors are used to transduce 100,000 host cells, the multiplicity of infection is 10. The use of this term is not limited to events involving transduction, but instead encompasses introduction of a vector into a host by methods such as lipofection, microinjection, calcium phosphate precipitation, and electroporation.

“Nucleic acid sequence” as used herein refers to an oligonucleotide, or polynucleotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules.

“Nucleic acid sequence” as used herein refers to an oligonucleotide, or polynucleotide, and fragments or portions thereof, and to DNA or RNA of genomic or synthetic origin which may be single- or double-stranded, and represent the sense or antisense strand. “Nucleic acid molecule” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

The phrase “pharmaceutically acceptable carrier” is used to mean any of the standard pharmaceutically acceptable carriers. Examples include, but are not limited to, phosphate buffered saline, physiological saline, and water.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by subcutaneous injection, intravenous injection, by subcutaneous infusion or intravenous infusion, for example by pump. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. For oral administration of peptides, techniques such of those utilized by, e.g., Emisphere Technologies well known to those of skill in the art and can routinely be used.

The pharmaceutical compositions of the present invention may be manufactured in a manner that is itself known, e.g., by means of conventional mixing, dissolving, granulating, dragee-making, levigating, spray drying, emulsifying, encapsulating, entrapping or lyophilizing processes.

Pharmaceutically acceptable carriers are well known to those skilled in the art and include, but are not limited to, 0.01-0.1M and preferably 0.05M phosphate buffer, phosphate-buffered saline, or 0.9% saline. Additionally, such pharmaceutically acceptable carriers may include, but are not limited to, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

Pharmaceutical formulations for parenteral administration include aqueous solutions of the active compounds in water-soluble form. Additionally, emulsions and suspensions of the active compounds may be prepared as appropriate oily injection mixtures. Suitable lipophilic solvents or vehicles include fatty oils such as sesame oil, or synthetic fatty acid esters, such as ethyl oleate or triglycerides, liposomes or other substances known in the art for making lipid or lipophilic emulsions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, trehalose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl-cellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

“Subject” shall mean any animal, such as a primate, mouse, rat, guinea pig or rabbit. In the preferred embodiment, the subject is a human.

“Therapeutically effective amount” means an amount sufficient to treat a subject afflicted with a disorder or a complication associated with a disorder. For example, the term “therapeutically effective amount” may refer to that amount of a compound or preparation that successfully prevents the symptoms of hepatic fibrosis and/or reduces the severity of symptoms. The effective amount of a therapeutic composition may depend on a number of factors, including the age, immune status, race, and sex of the subject and the severity of the fibrotic condition and other factors responsible for biologic variability.

Regulatory elements may be tissue specific or cell specific. The term “tissue specific” as it applies to a regulatory element refers to a regulatory element that is capable of directing selective expression of a nucleotide sequence of interest to a specific type of tissue (e.g., liver) in the relative absence of expression of the same nucleotide sequence of interest in a different type of tissue (e.g., lung).

Tissue specificity of a regulatory element may be evaluated by, for example, operably linking a reporter gene to a promoter sequence (which is not tissue-specific) and to the regulatory element to generate a reporter construct, introducing the reporter construct into the genome of an animal such that the reporter construct is integrated into every tissue of the resulting transgenic animal, and detecting the expression of the reporter gene (e.g., detecting mRNA, protein, or the activity of a protein encoded by the reporter gene) in different tissues of the transgenic animal. The detection of a greater level of expression of the reporter gene in one or more tissues relative to the level of expression of the reporter gene in other tissues shows that the regulatory element is “specific” for the tissues in which greater levels of expression are detected. Thus, the term “tissue-specific” (e.g., liver-specific) as used herein is a relative term that does not require absolute specificity of expression. In other words, the term “tissue-specific” does not require that one tissue have extremely high levels of expression and another tissue have no expression. It is sufficient that expression is greater in one tissue than another. By contrast, “strict” or “absolute” tissue-specific expression is meant to indicate expression in a single tissue type (e.g., liver) with no detectable expression in other tissues.

The term “cell type specific” as applied to a regulatory element refers to a regulatory element which is capable of directing selective expression of a nucleotide sequence of interest in a specific type of cell in the relative absence of expression of the same nucleotide sequence of interest in a different type of cell within the same tissue. The term “cell type specific” when applied to a regulatory element also means a regulatory element capable of promoting selective expression of a nucleotide sequence of interest in a region within a single tissue.

Cell type specificity of a regulatory element may be assessed using methods well known in the art (e.g., immunohistochemical staining and/or Northern blot analysis). Briefly, for immunohistochemical staining, tissue sections are embedded in paraffin, and paraffin sections are reacted with a primary antibody specific for the polypeptide product encoded by the nucleotide sequence of interest whose expression is regulated by the regulatory element.

“Transduction” is used to refer to the introduction of genetic material into a cell by using a viral vector.

As used herein a “transduced cell” results from a transduction process and contains genetic material it did not contain before the transduction process, whether stably integrated or not. As used in some prior art, but not as used herein, “transduced cells” may refer to a population of cells which has resulted from a transduction process and which population includes cells containing the genetic material and cells not containing the genetic material, whether stably integrated or not.

Transfection refers to the introduction of genetic material into a cell without using a viral vector. Examples of transfection include insertion of “naked” DNA or DNA in liposomes, that is without a viral coat or envelope.

“Treating” a disorder shall mean slowing, stopping or reversing the progression of the disorder and/or a related complication. In the preferred embodiment, “treating” a disorder means reversing the disorder's progression, ideally to the point of eliminating the disorder itself. As used herein in this context, “ameliorating” and “treating” are equivalent.

As used herein, “vector” shall mean any nucleic acid vector known in the art. Such vectors include, but are not limited to, plasmid vectors, cosmid vectors and bacteriophage vectors. For example one class of vectors utilizes DNA elements which are derived from animal viruses such as animal papilloma virus, polyoma virus, adenovirus, vaccinia virus, baculovirus, retroviruses (RSV, MMTC or MoMLV), Semliki Forest virus or SV40 virus.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “integrating vector” refers to a vector whose integration or insertion into a nucleic acid (e.g., a chromosome) is accomplished via an integrase. Examples of “integrating vectors” include, but are not limited to, retroviral vectors, transposons, and adeno associated virus vectors.

“Viral vector” is used herein to mean a vector that comprises all or parts of a viral genome which is capable of being introduced into cells and expressed. Such viral vectors may include native, mutant or recombinant viruses. A viral vector may be modified to express a gene of interest. Such viruses may have an RNA or DNA genome. Examples of suitable viral vectors include retroviral vectors (including lentiviral vectors), adenoviral vectors, adeno-associated viral vectors and hybrid vectors. Vectors that may be used include, but are not limited to, those derived from recombinant bacteriophage DNA, plasmid DNA or cosmid DNA. For example, plasmid vectors such as pcDNA3, pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors.

Alternatively, recombinant virus vectors including, but not limited to, those derived from viruses such as herpes virus, retroviruses, vaccinia viruses, adenoviruses, adeno-associated viruses or bovine papilloma viruses plant viruses, such as tobacco mosaic virus and baculovirus may be engineered.

As used herein, the term “integrated” refers to a vector that is stably inserted into the genome (i.e., into a chromosome) of a host cell.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a double-stranded provirus) into the genome of the host cell. The term “retrovirus” encompasses Oncovirinae (e.g., Moloney murine leukemia virus (MoMOLV), Moloney murine sarcoma virus (MoMSV), and Mouse mammary tumor virus (MMTV), Spumavirinae, and Lentivirinae (e.g., Human immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells which are susceptible to infection by the retrovirus. Through well known genetic manipulations, the replicative capacity of the retroviral genome can be destroyed. The resulting replication-defective vectors can be used to introduce new genetic material to a cell but they are unable to replicate. A helper virus or packaging cell line can be used to permit vector particle assembly and egress from the cell. Such retroviral vectors comprise a replication-deficient retroviral genome containing a nucleic acid sequence encoding at least one gene of interest (i.e., a polycistronic nucleic acid sequence can encode more than one gene of interest), a 5′ retroviral long terminal repeat (5′ LTR); and a 3′ retroviral long terminal repeat (3′ LTR).

The term “pseudotyped retroviral vector” refers to a retroviral vector containing a heterologous membrane protein. The term “membrane-associated protein” refers to a protein (e.g., a viral envelope glycoprotein or the G proteins of viruses in the Rhabdoviridae family such as VSV, Piry, Chandipura and Mokola) which are associated with the membrane surrounding a viral particle; these membrane-associated proteins mediate the entry of the viral particle into the host cell. The membrane associated protein may bind to specific cell surface protein receptors, as is the case for retroviral envelope proteins or the membrane-associated protein may interact with a phospholipid component of the plasma membrane of the host cell, as is the case for the G proteins derived from members of the Rhabdoviridae family.

As used herein, the term “adeno-associated virus (AAV) vector” refers to a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences.

AAV vectors can be constructed using recombinant techniques that are known in the art to include one or more heterologous nucleotide sequences flanked on both ends (5′ and 3′) with functional AAV ITRs. In the practice of the invention, an AAV vector can include at least one AAV ITR and a suitable promoter sequence positioned upstream of the heterologous nucleotide sequence and at least one AAV ITR positioned downstream of the heterologous sequence. A “recombinant AAV vector plasmid” refers to one type of recombinant AAV vector wherein the vector comprises a plasmid. As with AAV vectors in general, 5′ and 3′ ITRs flank the selected heterologous nucleotide sequence.

AAV vectors can also include transcription sequences such as polyadenylation sites, as well as selectable markers or reporter genes, enhancer sequences, and other control elements which allow for the induction of transcription. Such control elements are described above.

As used herein, the term “AAV virion” refers to a complete virus particle. An AAV virion may be a wild type AAV virus particle (comprising a linear, single-stranded AAV nucleic acid genome associated with an AAV capsid, i.e., a protein coat), or a recombinant AAV virus particle (described below). In this regard, single-stranded AAV nucleic acid molecules (either the sense/coding strand or the antisense/anticoding strand as those terms are generally defined) can be packaged into an AAV virion; both the sense and the antisense strands are equally infectious.

As used herein, the term “recombinant AAV virion” or “rAAV” is defined as an infectious, replication-defective virus composed of an AAV protein shell encapsidating (i.e., surrounding with a protein coat) a heterologous nucleotide sequence, which in turn is flanked 5′ and 3′ by AAV ITRs. A number of techniques for constructing recombinant AAV virions are known in the art (See, e.g., U.S. Pat. No. 5,173,414; WO 92/01070; WO 93/03769; all of which are incorporated herein by reference).

Suitable nucleotide sequences for use in AAV vectors (and, indeed, any of the vectors described herein) include any functionally relevant nucleotide sequence. Thus, the AAV vectors of the present invention can comprise any desired gene that encodes an antioxidant gene (e.g., STAP and catalase) having the desired biological or therapeutic effect of preventing or reversing liver cirrhosis.

By “adeno-associated virus inverted terminal repeats” or “AAV ITRs” is meant the art-recognized palindromic regions found at each end of the AAV genome which function together in cis as origins of DNA replication and as packaging signals for the virus. For use with the present invention, flanking AAV ITRs are positioned 5′ and 3′ of one or more selected heterologous nucleotide sequences and, together with the rep coding region or the Rep expression product, provide for the integration of the selected sequences into the genome of a target cell.

The nucleotide sequences of AAV ITR regions are known (See, e.g., Kotin, Human Gene Therapy 5:793-801 [1994]; Bems, K. I. “Parvoviridae and their Replication” in Fundamental Virology, 2nd Edition, (B. N. Fields and D. M. Knipe, eds.) for the AAV-2 sequence. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAVX7, etc. The 5′ and 3′ ITRs which flank a selected heterologous nucleotide sequence need not necessarily be identical or derived from the same AAV serotype or isolate, so long as they function as intended, i.e., to allow for the integration of the associated heterologous sequence into the target cell genome when the rep gene is present (either on the same or on a different vector), or when the Rep expression product is present in the target cell.

Integrating viral vectors are herein defined as those which result in the integration of all or part of their genetic material into the cellular genome. They include retroviral vectors and AAV vectors. They also include hybrid vectors such as adenoviral/retroviral vectors and adenoviral/AAV vectors. However, vectors that replicate stably as episomes can also be used. It is also desired that the vector can be produced in cell lines to a high titre, in a cost-effective manner, and have minimal risk for patients, for example not giving rise to replication competent virus.

This invention is illustrated in the Experimental Details section which follows. This section is set forth to aid in an understanding of the invention but is not intended to, and should not be construed to limit in any way, the invention as set forth in the claims which follow thereafter.

Experimental Details I

A. Synopsis

Cirrhosis is one of the most common causes of mortality in many countries. It affects more than 5% of the population worldwide, especially adults during their most productive years. Here we demonstrated that majority of primary stellate cells (>60%) can be transduced with rAAV/CAG-STAP particles (MOI: 1/1000) in vitro. In rats, a single injection with rAAV/CAG-STAP two weeks prior to treatment with CCl₄ for 8 consecutive weeks resulted in significant prevention of liver cirrhosis. Both levels of ALT and AST in the rats transduced with rAAV/CAG-STAP (rat or human) were very close to rats that were not transduced and not treated with CCl₄. In contrast, high ALT and AST levels were observed in CCl₄ treated rats which had been transduced with rAAV/CAG-EGFP or treated with PBS. Rats transduced with rAAV/CAG-STAP (rat or human) particles prior to induction with CCl₄ resulted in not only protection of the liver architecture but also maintenance of hepatic functions.

Transduction of STAP suppressed α-SMA, collagen I, and TGF-β, a major factor stimulating stellate cell fibrogenic activity, inhibited fibrogenesis and hepatocyte apoptosis, and improved the survival rates with this severe illness.

Transduction of STAP also resulted in the reverse of rat liver cirrhosis resulting from CCl₄ treatment. After treatment with rAAV/CAG-STAP particles for 4 weeks post CCl₄-induced liver damage, levels of ALT and AST decreased dramatically to nonpathological levels. Characterization of rAAV/CAG-human STAP might eventually be translated into a useful clinical trial of gene therapy for treatment of patients with progressive liver cirrhosis.

B. Methods

Animals: Young adult male Sprague-Dawley (SD) rats, weighing around 120 grams, were housed at a constant temperature and supplied with laboratory chow and water ad libitum. All studies were conducted under a research protocol approved by the Hong Kong SAR Government's Department of Health and the University of Hong Kong Animal Ethics Committee. All pathogen-free male SD rats except non-CCl₄ treated controls were administered with 0.5 ml/kg CCl₄ mixed with olive oil to a final concentration of 50% (vol/vol) subcutaneously twice a week for 8 weeks. For the prevention studies, the following groups were studied (n=10 rats/group): rats transduced with 3×10¹¹ rAAV/CAG-STAP (rat or human) particles/animal two weeks prior to treatment with CCl₄; rats transduced with 3×10¹¹ rAAV/EGFP two weeks prior to treatment with CCl₄; rats treated with PBS two weeks prior to treatment with CCl₄; and non-transduced and no CCl₄ treatment rats. One day after the final injection, rats were anesthetized by diethylether and the peritoneal cavity was opened. Removal and processing of tissue were carried out as previously described (Xu et al, 2003, in press). Liver tissues samples were stored at −80° C. before analysis.

cDNA cloning and generation of recombinant AAV vectors: RNA from 100 mg of the liver tissues was extracted using Trizol® (Life Technologies). First-strand cDNA was synthesized using 5.0 μg of total RNA, which was primed with Oligo dt (0.5 μg, Promega®), then reverse-transcribed using SuperScript® II RNase H reverse transcriptase (150 U; Life Technologies) at 42° C. for 90 minutes. Duplicate reactions without SuperScript® II were used as the negative controls. Insulin oligonucleotide primers, In-1,5′-CAG CCT TTG TGA ACC AAC AC-3′ (SEQ ID NO:1) and In-2,5′-GCG TCT AGT TGC AGT AGT TC-3′ (SEQ ID NO:2) were used to generate product. Analysis of β-actin cDNA was an internal control for the PCR reactions. Primers for β-actin PCR were (A-1,5′-CTC TTC CAG CCT TCC TTC C-3′) (SEQ ID NO:3) and (A-2, 5′-GTC ACC TTC ACC GTT CCA G-3′) (SEQ ID NO:4). The cycling parameters were 5 minutes at 94° C., followed by 40 cycles of 1 minute of 60° C. and 1 minute at 72° C. After amplification, 5 μl of PCR products were separated by gel electrophoresis on a 2% agarose gel containing ethidium bromide solution (Life Technologies) and visualized with UV light. Rat STAP cDNA was cloned from SD rat liver tissues by PCR using two oligonucleotide primers 5′-ATG GAG AAA GTG CCG GGC GAC-3′(SEQ ID NO:5) 5′-TGG CCC TGA AGA GGG CAG TGT-3′ (SEQ ID NO:6). The open reading frame of cloned rat STAP cDNA was inserted into the EcoR1 and Not 1 sites of the rAAV construct containing the AAV-2 inverted terminal repeats (ITRs), a CAG promoter and the woodchuck hepatitis B virus post-transcriptional regulatory element (WPRE) to facilitate expression (Xu et al. Hepatology, 2003, in press; and Xu et al., 2001,).

Recombinant AAV vectors expressing STAP, EGFP and empty particles were packaged and heparin column purified as previously described (Svegliati-Baroni et al., 1999; Xu et al. Hepatology, 2003, in press).

AAV particles were generated by a three plasmid, helper-virus free, packaging method. Briefly, rAAV vectors and the helper pFd H22 were transfected into 293 cells using calcium phosphate precipitation. Cells were harvested 70 hours after transfection and lysed by incubation with 0.5% deoxycholate in the presence of 50 units/ml benzonase (Sigma) for 30 minutes at 37° C. After centrifugation at 5000 g, the lysate was filtered through a 0.45 μm Acrodisc syringe filter to remove any particulate matter. The rAAV particles were isolated by heparin affinity column chromatography. The peak virus fraction was dialyzed against 100 mM NaCl, 1 mM MgCl2 and 20 mM sodium mono- and di-basic phosphate, pH 7.4. An aliquot was subjected to quantitative PCR analysis (AB Applied Biosystem) to quantify the genomic titer. A modified dot-blot protocol was used to perform the PCR Taqman assay, whereby AAV was serially diluted, and sequentially digested with DNAse I and Proteinase K. Viral DNA was extracted twice with phenol-chloroform to remove proteins, and then precipitated with 2.5 equivalent volumes of ethanol. A standard amplification curve was established at a range from 10² to 10⁷ copies, and the amplification curve corresponding to each initial template copy number was obtained. Viral particles were reconfirmed by commercial analysis kit (Progen, Germany). The viral particles were stored at −80° C. prior to animal experiments.

The titers of all vector stocks were measured by ELISA (Progen, Germany). In addition, titers of rAAV/CAG-STAP (rat and human) and rAAV/CAG-EGFP vectors were reconfirmed by an ABI Prism 7700™ Sequence Detection System.

Stellate cell isolation and culture: Preparation of hepatic stellate cells from non-transduced and rats untreated with CCl₄ and fibrotic rats was carried out as previously described (Kawada et al, 2001). Stellate cells isolated from non-transduced and no CCl₄ treatment rats or fibrotic livers were referred to as quiescent or in vivo activated stellate cells, respectively. An identical set of stellate cells or hepatocytes were transduced with rAAV viral particles at multiplicity of infection (MOI) ratio of 1:200. STAP gene expression was determined by western blot and immunochemistry (Kawada et al., 2001). 200 μM ascorbic acid and 10 μM FeNTA (final concentrations) was added to the cells to induce lipid peroxidation 48 hours after transduction. Markers of lipid peroxidation, such as MDA and 4-HNE were determined using the LPO-586™ kit (CalBiochem®, USA), while the cytotoxic effects of arachidonic acid were estimated by the MTT assay. MTT [3-(4,5-dimethylthiazol-2-yl)-2,5-diphenyl-2H-tetrazolium bromide] assay: add 50 μl of MTT (2 mg/ml). To each well of microtitre plate using the multichannel pipette. Incubate plates for 4 hours at 37° C. and 5% CO₂. Flick media and MTT from each plate into discard bowl and tip down sink. Add 150 μl of DMSO to each plate using multichannel pipette. Place plates in plate reader and read at 595 nm within 10 minutes of adding the DMSO. After stellate cells were transduced with rAAV/CAG-STAP particles, the stellate cells were divided into two groups. Lipid peroxidation was induced in one group but not the other in order to determine the efficiency of STAP in scavenging of radical-derived organic peroxides.

Electrophoretic gel mobility shift assay (EMSA): EMSAs are employed to demonstrate activation and translocation of proteins that bind to specific consensus DNA sequences. Binding sites for the AP-1 protein complex, 5′-AGC ATG AGT CAG ACA CCT CTT GGC-3′ (SEQ ID NO:7); or for the NK-kB protein complex, 5′-AGT TGA GGG GAC TTT CCC AGG C-3′ (SEQ ID NO:8); or for Egr protein complex, 5′-GGA TCC AGC GGG GGC GAG CGG GGC GA-3′(SEQ ID NO: 9); or for CEBP protein complex, 5′-TGC AGA TTG CGC AAT CTG CA-3′ (SEQ ID NO:10) were labeled using T4 polynucleotide kinase (Boehringer-Mannheim) and [γ³²P] ATP (4000 Ci/mmol, ICN Costa Mesa, Calif., USA). For competition studies, unlabelled AP-1 or NFkB, Egr and CEBP in 10-100 folds excess were included in the reaction mixture. After incubation of nuclear protein (5 μg) with 0.5 ng of labeled probe, the reaction mixture was resolved on a non-denaturing polyacrylamide gel. The gel was dried, autoradiographed, and radioactivity was measured with Phospho Imager™ (Bio-Rad®, USA). Supershift assays were performed with affinity purified, polyclonal antibody to p65 (Santa Cruz Biotechnology®, Santa Cruz, Calif.). For supershift assays, nuclear extracts were incubated with labeled probe as above, then incubated for an additional hour with 1.0 μg of the antibody.

Northern blotting: Northern blotting analysis was conducted as previously described (Ueki et al., 1999). Briefly, total cellular RNA was extracted from liver tissue with 1 ml of RNA-STAT-60™ (Tel-Test, Inc, Friendswood, Tex.) per-100 mm dish, following the manufacturer's instructions. Total RNA was separated by gel electrophoresis on an agarose gel and transferred to a Zeta-probe® GT nylon membrane (Bio-Rad® Laboratories, Richmond, Calif., USA). A DNA segment was cut from AAV/CAG-STAP plasmid and was labeled with [³²P] dCTP using random primer labeling kit (Gibco-BRL) and used for hybridization probes. Hybridization signals were detected using Biomax MS™ autoradiography film (Eastman Kodak Co., Rochester, N.Y.) and quantitated using a Bio-Rad GS-250 PhosphoImager™ (Bio-Rad, Hercules, Calif.). Northern analysis of and hybridization conditions for TGF-β1, TGF-α, TIMP-1, type-3 and type 4, MMP-2, and fibrinogen mRNA were carried out as previously described (Ueki et al., 1999; Nieto et al., 2001; and Kawada et al., 2001). The detection of hybridized cRNA probes were performed using 5-bromo-4-chloride-3-indolyl phosphate and nitroblue tetrazolium (Roche Molecular Biochemicals).

In situ hybridization: Liver slices were fixed for 7 minutes in 4% formaldehyde and washed in PBS for 3 minutes, 2×SSC for 10 minutes. The sections were hybridized at 37° C. for 24 hours in a mixture containing 4×SSC, 10% dextran sulfate, 1× Denhardt's solution, 2 mM EDTA, 50% deionised formamide, and 500 μg/ml herring sperm DNA. The slices were hybridized with DIG-labeled antisense cRNA. The labeling procedure was followed according to the DIG RNA labeling kit (Boehringer). The negative controls were hybridized with DIG-labeled sense cRNA. High stringency post-hybridization washes were performed in 60% formamide in 0.2×SSC at 37° C. for 15 minutes and in 2×SSC at room temperature for 10 minutes. Hybridization was detected by DIG immunological detection kit (Boehringer).

RT-PCR analysis for determination of gene expression induced by STAP: Total RNA was isolated from frozen tissue using RNAzol B. mRNA expression in each sample was determined by reverse transcription-polymerase chain reaction using GeneAmp® RNA PCR Core kit (PerkinElmer Life Science). The following primers were used: c-MET: 5′-GCA CCC CAA AGC TGG TAA TA-3′ (forward) (SEQ ID NO:11), 5′-CCG GTT GAA CGA TCA CTT TT-3′ (reverse) (SEQ ID NO:12); HGF: 5′-CGA GCT ATC GCG GTA AAG AC-3′ (forward) (SEQ ID NO:13), 5′-GGT GGT TCC CCT GTA ACC TT-3′ (reverse) (SEQ ID NO:14); Procollagen α type-1: 5′-TAC TAC CGG GCC GAT GAT GC-3′ (forward) (SEQ ID NO:15), 5′-TCC TTG GGG TTC GGG CTG ATG TA-3′(reverse) (SEQ ID NO:16); procollagen III: 5′-CCC CTG GTC CCT GCT GTG G-3′(forward) (SEQ ID NO:17), 5′-GAG GCC CGG CTG GAA AGA A-3′ (reverse) (SEQ ID NO:18); MMP-13: 5′-AGC TTG GCC ACT CCC TCG GTC TGT G-3′ (forward) (SEQ ID NO:19), 5′-GTC TCG GGA TGG ATG CTC GTA TGC-3′ (reverse) (SEQ ID NO:20); TGF-β1: 5′-TAT AGC AAC AAT TCC TGG CG-3′ (forward) (SEQ ID NO:21) and 5′-TGC TGT CAC AGG AGC AGT G-3′ (reverse) (SEQ ID NO:22); Tl1: 5′-CCA CAG ATA TCC GGT TCG CCT ACA-3′ (forward) (SEQ ID NO:23), 5′-GCA CAC CCC ACA GCC AGC ACT A-3′(reverse) (SEQ ID NO:24); WPRE: 5′-GCT AAA GAT TCT TGT ATA AAT CCT GGT TGC TGT CT-3′ (forward) (SEQ ID NO:25), 5′-GCA TCT CGA GGA AGG GAC GTA GCA GAA GAA C-3′ (reverse) (SEQ ID NO:26); Zf9: 5′-ACA ACC AGG AAG ACC TGT GG-3′ (forward) (SEQ ID NO:27), 5′-TGC TTT CAA GTG GGA GCT TT-3′ (reverse) (SEQ ID NO:28); and G3PDH: 5′-CCC TTC ATT GAC CTC AAC TAC ATG G-3′ (forward) (SEQ ID NO:29), 5′-CAT GGT GGT GAA GAC GCC AG-3′ (reverse) (SEQ ID NO:30). The receptor for hepatic growth factor (HGF) is a tyrosine kinase receptor encoded by c-met. Zf9 is a member of the Kruppel-like family of transcription factors that is induced in the well-defined, biologically important context of hepatic stellate cell activation. The modular structure of Zf9 has several interesting features including interaction with a promoter containing TATA box, that of collagen α1(1). G3PDH was used as an internal control. Analysis of the supression and/or inhibition of transcription factors during liver cirrhosis such as Sp-1, Zf-9/KLF6, JNK and p38 during liver cirrhosis were performed (Mendelson et al.; 1996).

TUNEL staining: Cell sensitivity to rAAV/EGFP or rAAV/CAG-STAP was assayed using the following procedure as in situ Cell Death Detect Kit™ (Roche Molecular Biochemicals). Serial sections of 8 μm thickness were prepared from liver tissues that had been fixed in 4% paraformaldehyde and embedded in paraffin (Xu et al., 2003, in press). Briefly, fixed sections were dewaxed and rehydrated and then permeabilized with a solution of 0.1% Trition-X100 and 0.1% sodium citrate. After blocking for 10 minutes in equilibration buffer, the reaction buffer containing TdT (terminal deoxynucleotidyl transferase) and fluorophore-labeled dUTP was added onto the section and incubated at 37° C. for 60 minutes. Reaction was terminated by transferring the slides into 1×SSC and incubating for 15 minutes at room temperature. Then, after a thorough washing in PBS, the sections were mounted in 3:1 Vectashield® DAPI and examined with confocal fluorescence microscope. Adjacent sections were counterstained with haematoxylin and eosin. The total number of apoptotic cells, in ten randomly selected fields, was counted. The apoptotic index (Al) was calculated as the percentage of positive staining cells. Al=number of apoptotic cells×100/total number of nucleated cells.

Immunohistochemical staining and analysis: The liver was postfixed in 30% sucrose in PBS and sections 20 μm in thickness were cut on a cryostat and thaw-mounted onto slides. Sections were rinsed three times with PBS containing 0.2% Triton-X100 prior to incubation in 1% H₂O₂ in methanol for 1 minute, rinsed three times in PBS, and then incubated with 4% defatted milk powder in PBS for 1 hour. After further PBS-Triton rinses, sections were incubated with the primary antibody overnight at room temperature. Sections were washed with PBS-Triton prior to a two hour incubation with secondary antibody, or immersed in propidium iodide solution (Sigma) for 5 minutes. The sections were then rinsed with PBS or distilled water before being mounted with Vectashield® (Vector La, Calif.). Immunofluorescent signals were captured using a Leica® 4d TCS confocal microscope, and images were processed using Adobe Photoshop® 5.0. Levels of TGF-B1, α-smooth muscle actin (α-SMA), proliferative cell nuclear antigen (PCNA), procollagen type I (PC-1), or NF-kβ (p50 and p65) were examined by immunohistochemistry. TGF-β1, endothelin-1, α-SMA were measured by ELISA and western blot. Production of polyclonal antibodies for STAP was carried out as previously report (Kawada et al., 2001, in press).

The PCNA labeling index was determined by counting more than 2,000 nuclei of hepatocytes in three different sections for each rat.

Masson's trichrome and HE staining: Paraffin-embedded sections were stained with Masson's trichrome and hematoxyllin-eosin. Liver cirrhosis was determined using computer image analysis techniques on Masson's trichrome-stained histologic slides. Histology examination was carried out to determine any pathological changes such as the collapse of parenchymal cells, the formation of regenerative nodules, distribution of fibrous septa, spread of reticulin fibers, the formation of thin fibrotic septa and a micro-nodular pattern of the parenchyma among the experimental groups.

Analysis of the differences among the area of fibrotic tissue, fibronectin, alpha-actin or collagen I, the activities of liver stellate cells, hemodynamic changes of portal and systemic blood pressures, the energy changes of liver, proteinase inhibitors, regeneration, serine proteinase and transgenic protein level as well as their overall effects on animal survival between the treated and the untreated are used to provide insights into function of STAP during liver cirrhosis. The synthesis of collagen was determined by a previously described procedure with some modification (Ueki et al., 1999)

The animals were divided into 5 groups i.e., rats transduced with 3×10¹¹ rAAV/CAG-EGFP particles/animal and then treated with CCl₄ for 8 consecutive weeks; rats treated with PBS only prior to treatment with CCl₄ for 8 consecutive weeks; rats transduced with 3×10¹¹ rAAV/CAG-rat STAP particles/animal for 2 weeks prior to treatment with CCl₄ for 8 weeks; rats transduced with 3×10¹¹ rAAV/CAG-human STAP particles/animal for 2 weeks prior to treatment with CCl₄ for 8 weeks; and normal rats (non-transduced and no CCl₄ treatment). Samples were processed as above. Blood samples were collected and serum was stored at −80° C. prior to analysis. Serial sections of 6 μm thickness were prepared from liver samples that have been frozen in liquid nitrogen, and stored at −80° C. Histology examination was carried out to determine any pathological changes such as the collapse of parenchymal cells, the formation of regenerative nodules, distribution of fibrous septa, spread of reticulin fibers, the formation of thin fibrotic septa and a micro-nodular pattern of the parenchyma among the experimental groups as previously described (Ueki et al., 1999). Lactase dehydrogenase (LDH) activity was measured and was regarded as an index of cytotoxicity. The lactate dehydrogenase assay kit (Sigma) was used to compare existence of cytotoxicity among the experimental groups. Mortality rates within each group were recorded.

Biochemical analysis: Serum albumin, bilirubin, aspartate transaminase (AST) (EC2.6.1.) and alanine transaminase (ALT) (EC 2.6.1.2) activities in rat blood were determined in Queen Mary Hospital, Hong Kong. LPO-586™ kit was used to measure the production of lipid peroxidation (a key consequence of oxidative stress) such as MDA and 4-HNE (CalBiochem, USA).

Whole liver homogenate was used to measure activity of proly hydroxylase (EC1.1.1.1) by techniques modified from Aguilar-Delfin et al. (1996). Catalase activity was measured as the decrease in absorbance at 240 nm due to H₂O₂ consumption. Catalase (EC 1.11.1.6) activity of STAP was determined spectrophotometrically by measuring the decrease of HO at 240 nm in 50 mM PBS buffer in the absence or presence of STAP. Fatty acid hydroperoxide peroxidase activity was determined according to Kharasch's method, slightly modified as described previously (Kawada et al., 2001). The total oxyradical scavenging capacity assay is based on the reaction between artificially generated oxyradicals and α-keto-γ-methiobutyric acid, which is oxidized to ethylene. The capacity of a sample to scavenge oxyradicals is quantified from its ability to inhibit ethylene formation relative to a control reaction containing no biological sample. The total oxyradical scavenging capacity (TOSC) assay is based on the reaction between artificially generated oxyradicals and α-keto-γ-meththiolbutyric acid (KMBA) which is oxidized to ethylene. For all samples, a specific TOSC value (referred to 1 mg of protein) was calculated by dividing the experimental TOSC values by the relative protein concentration contained in the assay.

Statistical analysis: Data are expressed as means±SEM and were analyzed by ANOVA with repeated measures and Tukey post-hoc tests using Systat® statistical software (Evanston, Ill.).

C. Results

STAP Gene Expression in the Liver

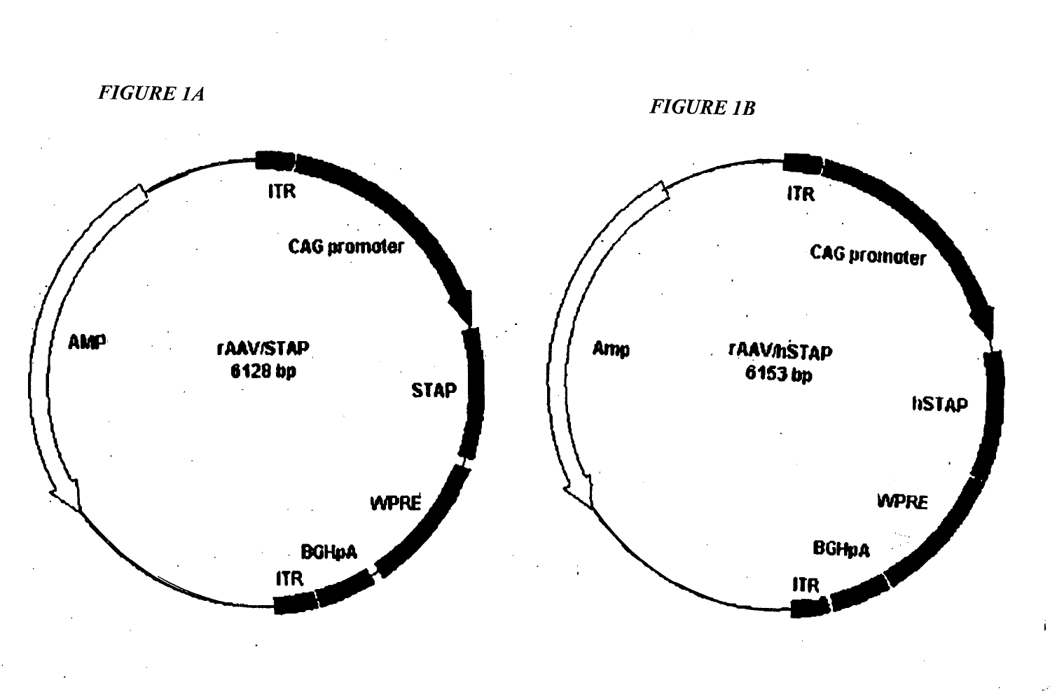

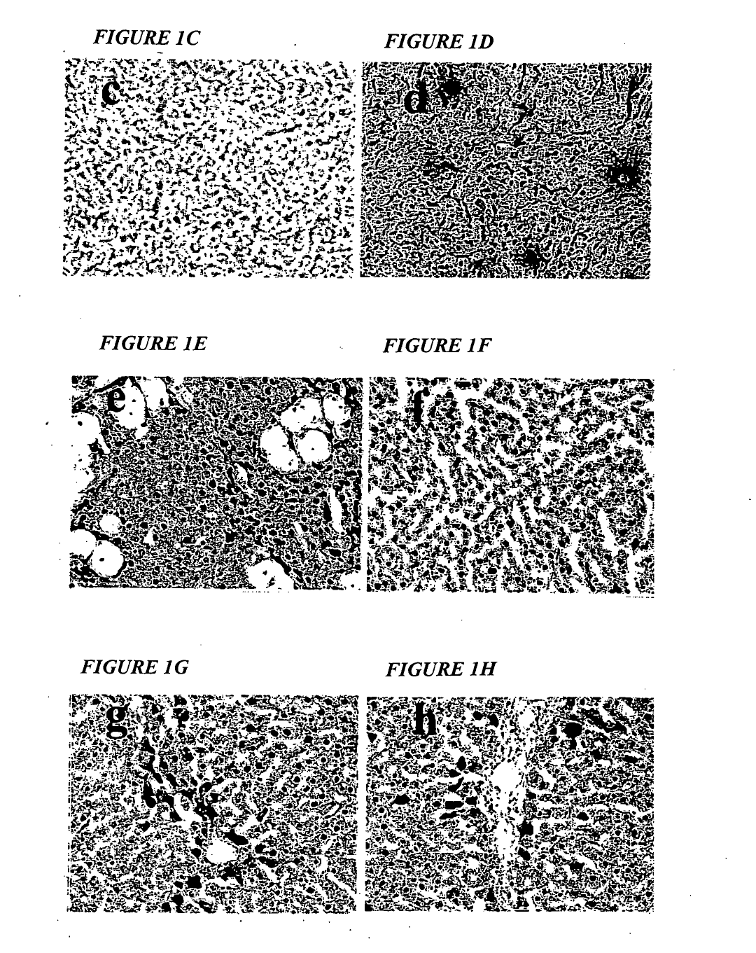

To assess ectopic expression of STAP in the liver, male SD rats were transduced with either rAAV/CAG-rat STAP particles containing the open reading frame having 570 base pairs coding 190 amino acids from the rat stellate cell activation-associated protein (Genbank Accession Number: NM_{—}130744; Kawada et al., 2001) (FIG. 1A) or rAAV/CAG-human STAP particles containing the open reading frame having 573 base pairs coding 191 amino acids of the human stellate cell activation-associated protein (Genbank Accession Number: AB057769; Ashahina et al., 2002) (FIG. 1B). The rAAV/CAG-rat STAP viral vector and the rAAV/CAG-human STAP viral vector was deposited with the China Center for Type Culture Collection (CCTCC), Wuhan, Hubei, China 430072, May 16, 2003 under the conditions of the Budapest Treaty and has been assigned the Patent Deposit Accession Numbers CCTCC-V200306 and CCTCC-V200305 respectively. The rats were sacrificed at 4 weeks after rAAV/CAG-STAP (human or rat) transduction. In situ hybridization revealed that, in contrast to the non-transduced rat group (treated with PBS) (FIG. 1C), the rAAV/CAG-STAP transduced rat group expressed STAP in the liver (FIG. 1D). To determine whether STAP mRNA was effectively translated into protein, STAP protein was measured in hepatic tissue by immuno-histochemistry. In contrast to both the rAAV/CAG-EGFP (FIG. 1E) and non-transduced rat groups (FIG. 1F), liver sections from rats transduced with either rAAV/CAG-rat STAP (FIG. 1G) or rAAV/CAG-human STAP (FIG. 1H) for 10 weeks showed strong STAP gene expression. These results demonstrate that the introduction of rat or human STAP by transduction with viral particles can increase transgenic STAP by can levels in these animals.

STAP Gene Expression in the Liver Prevented Hepatic Cirrhosis

To test the utility of STAP as a therapeutic gene, and in particular its potential for preventing exacerbated fibrosis, male SD rats were transduced with either rAAV/CAG-rat STAP (n=10 rats), rAAV/CAG-human STAP (n=10 rats), or rAAV/CAG-EGFP (n=10 rats) particles. Viral particles were delivered at a concentration of 3×10¹¹ particles/animal via portal vein injection two weeks prior to treatment with or without CCl₄ for 8 weeks. An additional group of rats (n=6) were treated with PBS only (non-transduced) prior to CCl4 treatment with or without CCl₄ for 8 weeks.

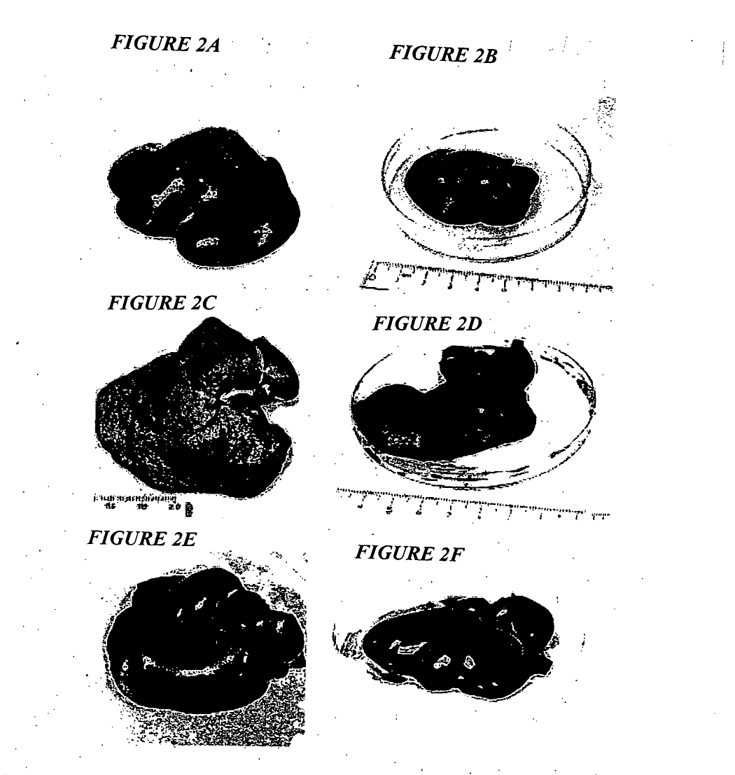

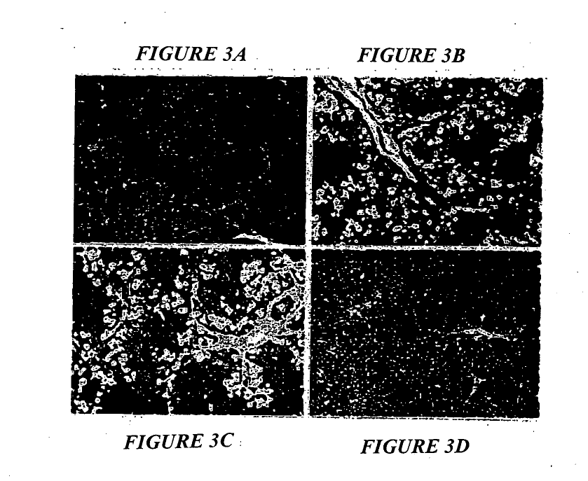

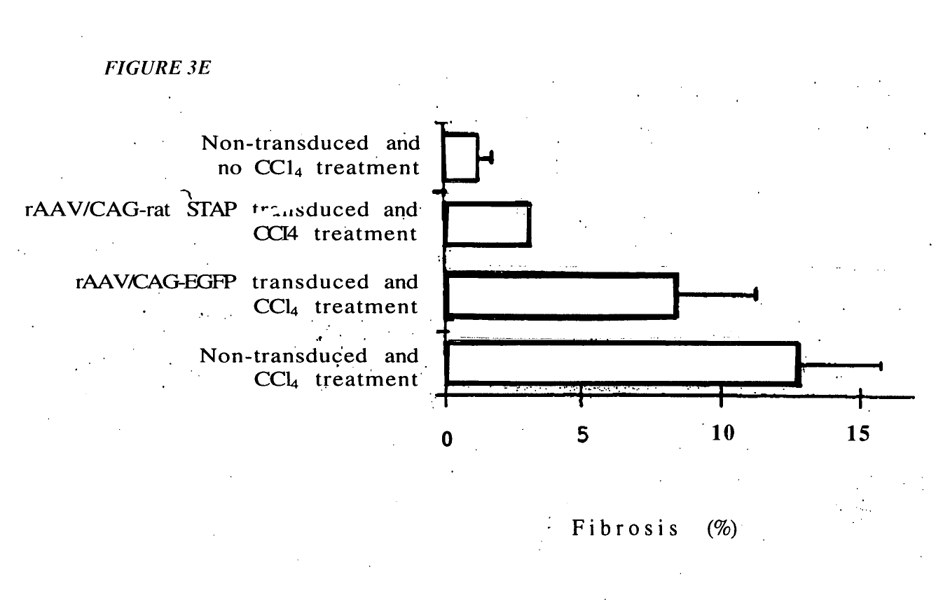

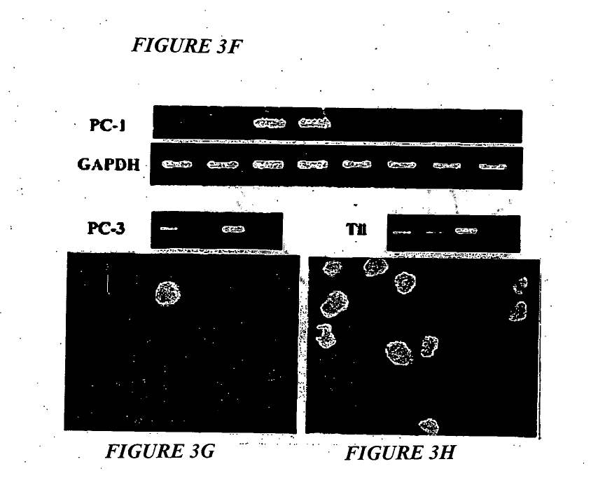

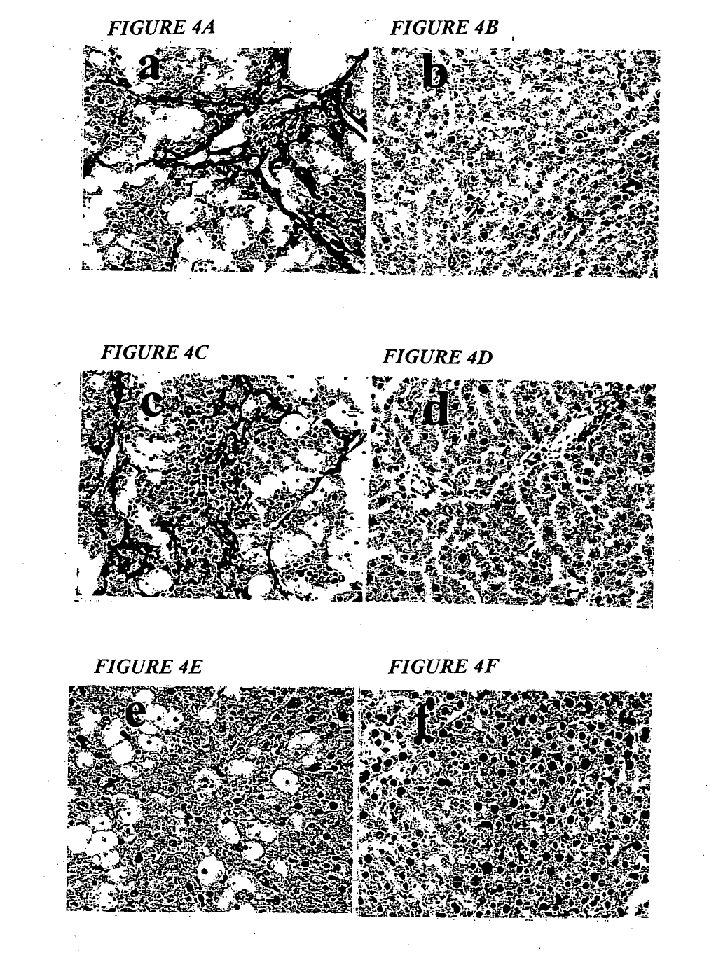

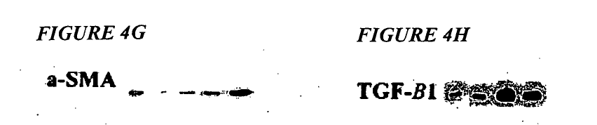

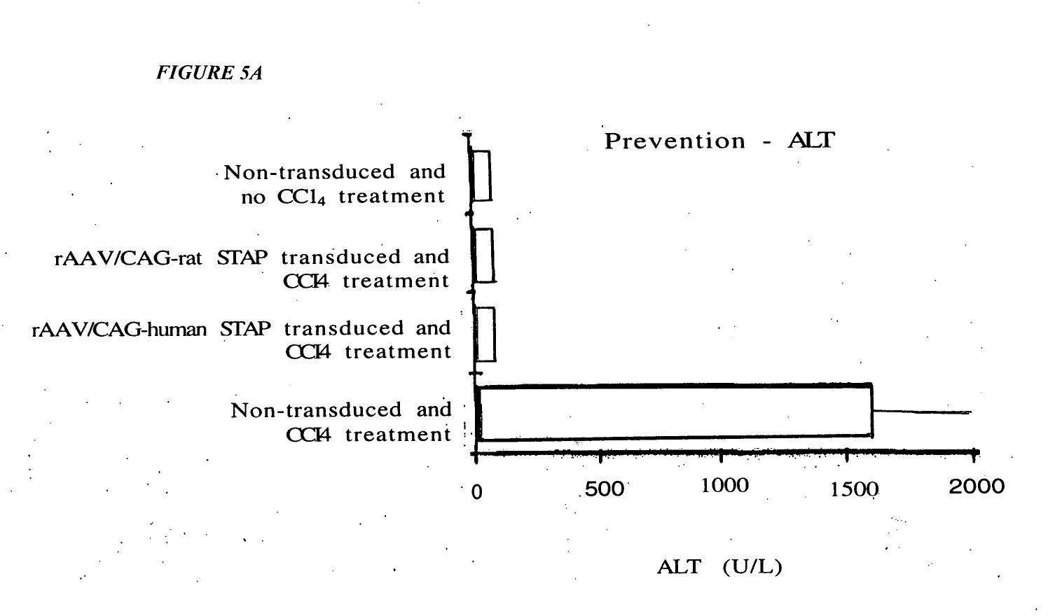

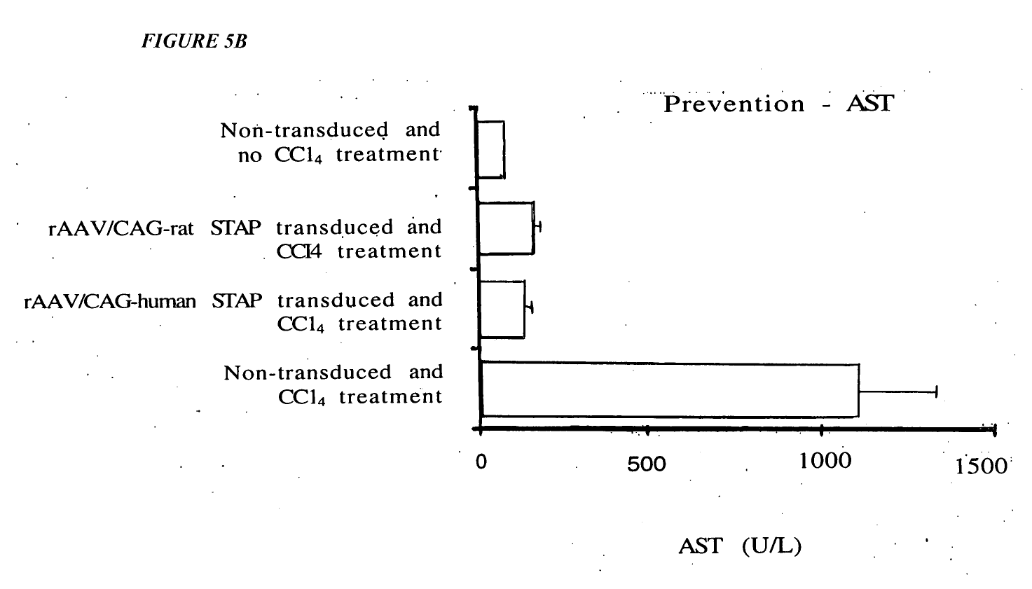

Hepatic architecture of the rats transduced with rAAV/CAG-rat STAP (FIGS. 2E and 2F; FIG. 3D) was similar to that of non-transduced and no CCl₄ treatment rats (FIGS. 2A and 2B; FIG. 3A). By contrast, the liver architecture became distorted in the non-transduced and rAAV/CAG-EGFP transduced groups after the eighth weekly administration of CCl₄. The distortion was marked by extensive fibrotic replacement (FIGS. 2C and 2D; FIGS. 3B and 3C), a micronodular pattern of the parenchyma throughout the livers of all rats (FIGS. 3B and 3C), and cessation of hepatocyte proliferation (FIG. 4E). The parenchymal cells collapsed, and regenerative nodules were formed, separated by fibrous septa. Reticulin fibers spread radially throughout the liver. The formation of thin fibrotic septa joining the central areas was observed, and a micronodular pattern of the parenchyma was evident in all rats. Assessment of fibrosis in the livers of all rats revealed that the index of collase I positive areas in the rats transduced with either with rAAV/CAG-rat STAP or rAAV/CAG-human STAP particles prior to treatment with CCl₄ was very close to that of non-transduced and no CCl₄ treatment rats, while the index of collases in non-transduced rats treated with CCl₄ or rAAV/CAG-EGFP transduced rats CCl₄ was twice as high (FIG. 3E). Fibrous connective tissue components in Glisson's sheath and pseudosoluble formations found in the cirrhosis of non-transduced rats or rAAV/CAG-EGFP transduced rats were inhibited by transduction with rAAV/CAG-STAP (human or rat). RT-PCR analysis of hepatic tissue from the rats treated with PBS prior to treatment with CCl₄ for 8 weeks showed that procollase I (PC-1) levels increased dramatically (FIG. 3F, lanes 1 and 2), while PC-1 levels of rats transduced with either rAAV/CAG-human STAP or rAAV/CAG-rat STAP particles prior to CCl₄ treatment were similar to those of non-transduced and no CCl₄ treatment rats (FIG. 3F, lanes 5 and 6; lanes 7 and 8; and lanes 3 and 4 respectively). Procollase III (PC-3) levels (FIG. 3G) and TII levels (FIG. 3H) also presented the same trends.

TGF-β1 has been identified as a major factor stimulating fibrogenic activity in stellate cells, a hallmark of human liver cirrhosis. After 8 weeks consecutive CCl₄ injury, increase of TGF-β1 level was found non-transduced rats (FIG. 4A). TGF-β1 was predominantly expressed in centrilobular areas and correlated with an enhanced number of α-smooth muscle antigen (α-SMA) (FIG. 4C) and desmin-positive cells (data no shown), which are both markers of activated stellate cells. TGF-β1 mRNA gene expression was reduced by the transduction of rAAV/CAG-rat STAP (FIG. 4H, lane 2). The TGF-β1 level was much higher in liver extracts from either rAAV/CAG-EGFP transduced rats or non-transduced rats (FIG. 4H, lane 4 and lane 1 respectively). Moreover, the hepatic stellate cells positive for desmin increased in the fibrotic regions of the cirrhosed livers of the treated group, and many of them were transformed into myofibroblast-like cells that specifically express α-SMA (FIGS. 4C and 4G). These data suggest that TGF-β1 induces the phenotypic transition of hepatic stellate cells to proliferating myofibroblast-like cells, which enhances the production of extracellular matrix components. TGF-β1 has been regarded as a potent growth inhibitor of epithelial and endothelial cells, including hepatocytes. To assess the over-expression of STAP on mitotic hepatocytes, the presence of mitotic hepatocytes was also assessed by immunohistochemical staining. The number of PCNA positive hepatocytes was much higher in the rAAV/CAG-rat STAP-transduced group (FIG. 4F) There was a substantial increase in the number of mitotic figures, binucleated hepatocytes and cells expressing PCNA.

To determine whether transgenic STAP can prevent apoptotic cell death caused by CCl₄ treatment, the apoptotic status of hepatocytes after transduction of the STAP gene and CCl₄ treatment was assessed. TUNEL staining revealed apoptotic cells were presented in the liver sections of all experimental groups. However, ectopic STAP gene expression prevented hepatocyte apoptosis induced by CCl₄ (FIG. 31). The numbers of apoptotic cells in the liver sections of rats transduced with either rAAV/CAG-rat STAP or rAAV/CAG-human STAP were similar to non-transduced and no CCl₄ treatment rats, while the number of apoptotic cells in the livers of non-transduced and CCl₄ treated rats were 2-4 folds higher (FIG. 3J). Taken together these data demonstrate that ectopic gene expression of STAP is sufficient to prevent liver cirrhosis (FIGS. 3D, 3I, 4B, 4D and 4F).

The effect of transgenic STAP expression on physiological functions was tested in order to ascertain its potential as a therapeutic gene to restore liver functions. Biochemical analysis showed that serum levels of alanine amino-transferase (ALT) and asparatate aminotransferrase (AST) response to CCl₄ were similar in both the rAAV/CAG-rat STAP (n=10) and rAAV/CAG-human STAP (n=10) groups, and were very close to non-transduced and no CCl₄ treatment group (n=10), suggesting that the liver functions in the groups treated with rAAV/CAG-STAP were not significantly affected by CCl₄ treatment. By contrast, a high serum level of ALT (FIG. 5A) and AST (FIG. 5B) was observed in rats either transduced with rAAV/CAG-EGFP (n=10) or treated with PBS (n=6). The transduction of rats with rAAV/CAG-STAP particles (human or rat) prior to treatment with CCl₄ resulted not only in the protection of the liver architecture but also in the restoration of hepatic functions.

Ectopic STAP Gene Expression Reversed Exacerbated Hepatic Fibrosis

Critical analysis of the various conditions characterized by cirrhosis allows the evaluation of the contribution of oxidative stress to pathogenesis with or without transduction with rAAV/CAG-STAP. To test the potential of STAP for the treatment of liver cirrhosis, forty animals were treated with CCl₄ for 8 weeks prior to transduction with rAAV/CAG-rat STAP, rAAV/CAG-human STAP, rAAV/CAG-EGFP or prior to treatment with PBS (n=10 rats/group). All animals were sacrificed 4 weeks later. Histochemistry revealed a similar trend as was observed in the prevention experiments. Biochemical analysis also showed similar trends to those observed in the prevention study (FIGS. 5C and 5D).

STAP Expression Reduced Oxidation Stress in Stellate Cells

The activation of one type of liver cell, the hepatic stellate cell (HSC), has long been considered as the central event in liver cirrhosis. Preventing HSC activation can slow down and even reverse cirrhosis. To ascertain whether HSC can be transduced with recombinant AAV vectors directly, stellate cells were isolated from non-transduced and untreated livers and cultured for 3 days, transduced with rAAV/CAG-STAP particles and then cultured for two days. Immunohistochemical results from in vitro study showed that over 60% of primary stellate cells can be directly transduced with recombinant AAV particles (FIG. 5F). Western blotting of extracts of primary stellate cells after transduction with rAAV/STAP particles for two days further confirmed this conclusion (data not shown). To test if STAP functions as an antifibrotic scavenger of peroxides during the progress of liver cirrhosis, primary stellate cells were cultured for 7 days and then aliquoted to different wells and kept at 37° C. in an incubator overnight. Stellate cells (n=3 wells) were transduced with rAAV/CAG-rat STAP, rAAV/CAG-human STAP or rAAV/CAG-EGFP particles (MOI 1:1000) for 48 hours. Oxidative stress of stellate cells was induced with Fe-NTA and arachidonic acid for 6 hours. Levels of 4-HNE fell markedly more in the groups transduced with rAAV/CAG-rat STAP and rAAV/CAG-human STAP than in the PBS treated or the rAAV/CAG-EGFP transduced groups (>20%)(data not shown). This clearly demonstrated that STAP acts as an antifibrotic scavenger of peroxides. STAP protein can catabolized hydrogen peroxide and lipid hydroperoxides, both of which have been shown recently to trigger stellate cell activation.

STAP Induced Changes in AP-1 Binding Activity

The response of stellate cells to injury represents a cellular program with a distinct temporal sequence involving both up- and down-regulation of gene expression. Analysis of gene expression in freshly isolated cells from a normal or injured liver provides an accurate profile of their behavor in vivo. RT-PCR on the total RNA extracted from the livers of the non-transduced rats or rats transduced with rAAV/CAG-STAP particles revealed that Zf9 expression and biosynthesis increased markedly in the non-transduced group treated with CCl₄ (FIG. 5G, lanes 3 and 4). Levels of Zf9 in both the rAAV/CAG-rat STAP transduced and rAAV/CAG-human STAP transduced groups were very similar to non-transduced and no CCl4 treatment group (FIG. 5G, lanes 7 and 8, lanes 5 and 6; and lanes 1 and 2 respectively). To explore the potential association between AP-1, induction of oxidation stress and changes in mRNA levels for c-jun following the rAAV/CAG-STAP transduction, c-jun and c-fos levels were measured (data not shown). It was then determined whether the transcriptional activation of the c-fos and c-jun resulted in the formation of a functional AP-1 complex. To accomplish this, an electrophoretic gel mobility shift assay was used to compare the ability of nuclear proteins, isolated from transduced, and non-transduced stellate cells, to bind to an AP-1 consensus sequence. Binding activity of nuclear extracts prepared from rAAV/CAG-STAP transduced rat livers to the oligo-nucleotide probe containing an AP-1 binding site was clearly reduced (data not shown).

STAP Prevented Increased Nuclear Levels of NK-kB in Response to Oxidative Stress

Activation of NF-kB binding is highly responsive to stress stimuli. Super-shift analysis of nuclear extracts prepared from activated HSC transduced with or without STAP vector for two days prior to exposure to ROS for 18 hours confirmed that the response of HSC to oxidative stress represents a cellular program with a distinct temporal sequence involving both up- and down-regulation of gene expression involving redox-sensitive transcription factor NF-kB. Furthermore, super-shift analysis with antibodies specific to the p50 subunits of NF-kB revealed that the mobility of the binding complexes was further retarded by the antibodies, indicating that p65/p50 heterodimers and possibly p50 homodimers accumulated in the nucleus following induction with F-NTA. However, binding activities of HSC transduced with STAP vectors were even lower than levels in untreated cells. These results suggest that over expression of STAP in HSC can block nuclear translocation of proteins that bind genomic kB elements in response to oxidative stress.

D. Discussion

The high transduction efficiency of both rAAV/CAG-rat STAP and rAAV/CAG-human STAP particles of hepatic stellate cells in vitro suggests that these recombinant AAV vector could be considered as an ideal delivery system to treat liver cirrhosis. Previous biochemical characterization of recombinant rat STAP revealed that STAP was a novel endogenous peroxidase exhibiting peroxidase activity toward hydrogen peroxide and linoleic acid hydroperoxide.

Evidence of the involvement of certain reaction free radicals or derived molecules in chronic pathologies was first considered. Particular attention was paid to the possible interference, by oxidative stress, with gene expression of fibrogenic tissue degeneration. Chronic liver damage with the pro-oxidant agent CCl₄ produces increased transcription and synthesis of TGF-β, in a process that is clearly limited to nonparenchymal cells (Poli et al., 1997). The direct correlation between oxidative stress and TGF-β expression and fibrogenic role comes from evidence that the up-regulation of TGF-β was in all cases paralleled by increased expression of the procollagen type I. Involvement of lipid peroxidation in the CCl₄ chronic liver damage model is supported by the increased production of malonaldehyde (MDA) and other more toxic carbonyl compounds such as 4-hydrooxyalkenals. Collagen type I co-localizes in areas positive for MDA and HNE protein adducts (Poli et al., 1997). A link between CCl₄ treatment induced lipid peroxidation, increased procollagen α-1 mRNA levels and collagen deposition in fibrotic livers has been established (Lee et al. 1995). It has been reported that the lipid peroxidation induced by CCl₄ treatment can be prevented by suitably supplementing the rat liver with vitamin E (Poli et al., 1997). The down-regulation of TGF-β1 expression in normal liver in the presence of a threefold increase in the tocopherol (vitamin E) concentration proves that redox reactions are also involved in the genetic regulation of this cytokine. TGF-β1 may play a key role during tissue repair and fibrogenesis (Poli et al., 1997; Friedman, 2000). This pleiotropic polypeptide has many effects on the extracellular matrix, including an ability to increase the amount of connective tissue. In response to treatment with CCl₄, transduction of stellate cells with rAAV/CAG-STAP particles, both in vitro and in vivo, suppressed TGF-β1 (a major factor stimulating stellate cell fibrogenic activity), inhibited fibrogenesis and hepatocyte apoptosis, and improved the survival rates. STAP can play a role as an anti-fibrotic scavenger of peroxides in the liver, as it completely abolished the over-expression of both TGF-β1 and collagen I, the key fibrogenic growth factor.

Marked oxidative disruption of cell structure and function is known to exert irreversible damage by various mechanisms. A variety of factors are up-regulated in activated stellate cells and are thought to contribute to the development of fibrosis in a highly orchestrated manner. The effect of oxidative stress on cytokine gene expression appears to be an important mechanism by which connective tissue deposition is promoted (Poli et al., 1997). Reactive oxygen species have been shown to induce the activation of at least two families of transcription factors: activator protein-1 (AP-1) and nuclear factor-kB (NF-kB). The AP-1 binding sequence is present in a number of eukaryotic genes, and it is activated through the interaction with homo- and heterodimers of the jun-fos nuclear protein family (Friedman, 2000; Whalen et al., 1999). The AP-1 transcription factor has been shown to be upregulated in response to oxidative stress resulting from CCl₄ treatment both in cell culture and in the intact rat. The transcription factor NF-kB is present in the cytosol as an inactive heterodimer complexed to an in inhibitor protein, which masks both nuclear localization signal and DNA binding portion. Translocation of NF-kB in response to most, but not all, stimuli involves an oxidant sensitive regulatory step (Poli et al., 1997; Whalen et al., 1999). Nuclear levels of NF-kB were significantly increased in the livers of CCl₄-treated rats due to increased oxidative stress as compared to NF-kB levels in the non-transduced and no CCl4 treatment rats or rats transduced rAAV/CAG-STAP particles. These results demonstrate that lipid peroxidation plays a role in activating HSC by an antioxidant sensitive pathway involving the redox-sensitive NF-kB transcription factor. Also, the oxidation-dependent activation of NF-kB and AP-1 in the rats treated with CCl₄ can be mediated and/or reversed by STAP expression.

The concept that gene expression is modulated by oxidant species is supported by the fundamental observations that (1) oxidative stress modulates the expression of genes encoding for cytokines at the transcriptional level (Mendelson et al., 1996), (2) lipid peroxidation upregulates the expression and synthesis of fibrogenic cytokines, and (3) aldehydic end products of lipid peroxidation enhance type I collagen synthesis by HSC (Parola et al., 1998). These events are initiated by the activation of transcription factors, leading to the mRNA expression of extracellular matrix matrices and tissue inhibitor of matrix metalloproteinase-1 and -2 (Bahr et al., 1999. A potential mechanism for the prevention of liver cirrhosis by rAAV/CAG-STAP is through inhibition of latent metalloproteinases (MMPs) complexed with TIMPs (tissue inhibitor of metalloproteinases). TIMP-1 (tissue inhibitor of metalloproteinases 1) expression is upregulated in activated HSC, and is therefore potentially an autocrine survival factor for HSC. The pattern of expression of TIMP-1 and TIMP-2 mRNA in the liver closely mirrored the appearance of pathology, suggesting that these genes might indeed be playing an important role. These MMPs are effector proteins downstream of urokinase-type plasminogen (uPA) in the matrix proteolysis cascade. It has been shown that expression of MMP-2 is increased in liver homogenates of rAAV/CAG-STAP transduced animals. MMP2 specifically degrades collagen type IV and other collagens to a lesser degree. However, amounts of active MMP-2 and MMP-2 species complexed with its specific inhibitor, TIMP-1 need to be quantitated.

Moreover, the degree of peroxidation, a key consequence of oxidative stress in which HNE plays a part, needs to be analyzed. In general, there is overproduction of reactive oxygen free radicals (ROS) and/or reactive nitrogen free radicals (RNS) during oxidative stress. Evidence of oxidative reaction is often associated with the onset of liver cirrhosis. NF-kB-binding sites are in the promoter region of GM-CSF, TNF-β1, IL-6 and growth factors relevant to inflammation. Gene activation of TGFβ-1, the most fibrogenic cytokine, and PDGF occurs through binding to the AP-1 site present on the long terminal repeat (Poli et al., 1997; Mari and Cederbaum, 2000).

Traditional pharmacological approaches to the treatment of human diseases have led to significant advances in health management. However, despite many major successes, no definitive cure for liver cirrhosis has yet been developed. Scavenging of radical-derived organic peroxides by STAP could be an adaptive reaction to normalize the cellular redox status during the cell activation. STAP could thus play a role as an antifibrotic scavenger of peroxides in the liver (Kawada et al., 2001). The potential application of gene therapy protocols to human hepatic cirrhosis depends on the successful and tissue-specific delivery of therapeutic genes to livers affected with extensive fibrosis. Therefore STAP might be an ideal therapeutic gene for liver cirrhosis prevention and treatment. Furthermore, HGF infusion into normal rat livers has been reported to stimulate hepatocyte proliferation only in the periportal areas (Lee, 1997; Salgado et al., 2000). In a rat cirrhosis model, a single i.v. administration of a replication-deficient adenoviral vector encoding a nonsecreted form of human uPA resulted in high production of functional uPA protein in the liver. This led to induction of collagenase expression and reversal of fibrosis with concomitant hepatocyte and improved liver function. uPA gene therapy might potentially be an effective strategy for treating cirrhosis in humans (Salgado et al., 2000). Neverthless, it has become increasingly clear in recent decades that the plasminogen activation systems, which include uPA, plasminogen activator inhibitor receptor (uPAR), and plasminogen activator inhibitors PAI-1 and PAI-2, play a very important role in the aggressiveness of cancer. Furthermore, the bleeding tendency of wild type uPA and the use of adenoviral vector as the gene delivery system limit the efficacy and safety of this approach (Salgado et al., 2000). Biochemical characterization of recombinant STAP revealed that STAP was a novel endogenous peroxidase exhibiting peroxidase activity toward hydrogen peroxide and linoleic acid hydroperoxide, suggesting that STAP acted as an antifibrotic scavenger of peroxides to prevent activation of HSC via multi-mechanism, and is a suitable therapeutic gene for cirrhosis therapy.

In summary, the studies described above demonstrate that transduction of rats with rAAV/CAG-STAP particles reduces levels of TGF-β1 and α-SMA, and prevents of CCl₄-induced liver cirrhosis. Transduction of STAP suppressed the expression of TGF-β, collagen I and α-SMA. A single dose of rAAV/CAG-STAP prevents and can reverse liver cirrhosis. Further characterization of rAAV/CAG-STAP could be translated into clinical trials and the development of a gene therapy treatment for patients with progressive liver cirrhosis.

Experimental Details II

A. Methods

cDNA cloning and generation of recombinant AAV vectors RNA from 100 mg of the liver tissues was extracted using Trizol (Life Tech.). First-stand cDNA was synthesized using 5.0 μg of total RNA, which was primed with Oligo dT (0.5 μg, Promega), then reverse-transcribed using SuperScript II RNase H⁻reverse transcriptase (150 U; Life Tech.) at 42° C. for 90 min. Duplicate reactions without SuperScript II were the negative controls. The cycling parameters were 5 min at 94° C., followed by 40 cycles of 1 min of 60° C. and 1 min at 72° C. After amplification, 5 μl of PCR products was electrophoresed on a 2% agarose gel (Life Tech) and visualized with UV light. STAP cDNA was cloned from SD rat liver tissues by PCR using a pair of primers 5′-ATG GAG AAA GTG CCG GGCGAC-3′,5′-CTA TGG CCC TGA AGA GGG CAG TGT-3′ for rat and for human respectively. The open reading frame of rat STAP cDNA was cloned into the EcoR1 and Not 1 sites of the rAAV construct containing the AAV-2 ITRs, a CAG promoter and the woodchuck hepatitis B virus post-transcriptional regulatory element (WPRE) to facilitate expression respectively. Recombinant AAV vectors expressing STAP, GFP and empty particles were packaged and heparin column purified. The rAAV viral genome titer was quantified by Real-time PCR using Taqman (Perkin-Elmer Biosystem, Calif.).

Stellate cell isolation and culture Preparation of hepatic stellate cells was according to published work. Briefly, liver was perfused first with a Ca²⁺/Mg²⁺-free solution and next with digestion for 15 minutes at 37° C. The softened liver was dispersed in solution with 0.05% collagenase, 0.02% pronase E and 0.005% Dnasel for 15 minutes at 37° C. The resulting suspension was washed by centrifugation (50 g, 5 min,) and the non-parenchymal cells were pelleted by centrifugation (450 g, 10 min, 20° C.). A stellate cell-enrich fraction was obtained by centrifugation on an 18% Nycodenz cushion (1400 g, 20 min, 20° C.) and washed two times by centrifugation (450 g, 10 min, 20° C.) and suspend in DMEM (GIBCO) supplemented with 10% fetal bovine serum (FBS). Cell purity was always more than 98% as assessed by immunocytochemistry detecting desmin. Lipid peroxidation insult was induced 48 hours after HSC were transduced with rAAV vectors (MOI 5×10⁴) by adding Fe-NTA and arachidonic acid to culture medium to final concentrations of 50 μM and 20 μM respectively. These experimental groups were designated HSC-control, HSC-rAAV/rSTAP, HSC-rAAV/hSTAP & HSC-rAAV/eGFP respectively. Lipid peroxides, including MDA and 4-HNE (a key consequence of oxidative stress), in the cell lysate and medium were determined by LPO-586 kit (CalBiochem, USA) at 0, 6 and 18 hours after peroxidation insult.

Western Blotting Tissues were excised, minced, and homogenised in protein lysate buffer. Protein samples (100 g) were resolved on 10% polyacrylamide SDS gels, and electrophoretically transferred to nitrocellulose Hybond C extra membranes (Amersham Life Science, England). After the membranes were blocked with 5% BSA, blots were incubated with specific primary Abs, followed by horseradish peroxidase-conjugated secondary antibodies, and developed by enhanced chemilumine-scence (Amersham International plc, England) and exposure to X-Ray film.

Animals Young adult male Sprague-Dawley rats, weighing around 120 grams, were housed at a constant temperature and supplied with laboratory chow and water ad libitum. All studies were conducted under a research protocol approved by the Hong Kong Government's Department of Health and the University of Hong Kong Animal Ethics Committee. For protection study, all pathogen-free male SD rats except the normal animals group were administered with 0.5 ml/kg CCl₄ mixed with olive oil to a final concentration of 50% (vol/vol) i.p. twice weekly for 8 weeks. The animals were divided into 5 groups (n=10): Group 1, Normal control, normal rats treated with PBS (Also the intraportal injection) only; Group 2, CCl₄-control, rats intraportal venous PBS injection two weeks prior to induction with CCl₄ for 8 consecutive weeks (chronic CCl₄ animal model); Group 3, 4 & 5, CCl₄-AAV/eGFP, CCl₄-AAV/rSTAP, CCl₄-AAV/hSTAP—rats transduced with 3×10¹¹ rAAV particles each of rAAV/EGFP, rAAV/rSTAP & rAAV/hSTAP per animal respectively two weeks prior to induction by CCl₄. For those animals for treatment study, forty animals induced with CCl₄ twice weekly for consecutive 8 weeks, then were injected with viral vectors respectively. The samples were stored at −80° C. before analysis. All viral vectors were delivered via portal vein.

Electrophoretic gel mobility shift Assay (EMSA) EMSA was employed to assess the abundance of transcription factors that bind to specific consensus DNA sequences for AP-1 and NF-kB. Twenty ng each of AP-1 protein complex (5′-AGC ATG AGT CAG ACA CCT CTT GGC-3′) and NF-κB protein complex (5′-AGT TGA GGG GAC TTT CCC AGG C-3′) consensus oligonucleotides (Santa Cruz) was labeled with 50Ci [32γP] ATP (4000 Ci/mmol, ICN Costa Mesa, Calif., USA) by T4 polynucleotide Kinase (Boehringer-Mannheim). For competition studies, unlabelled AP-1 or NF-kB and CEBP 5′-TGC AGA TTG CGC AAT CTG CA-3′ in 50 folds excess are included in the reaction mixture. After incubation of nuclear protein with labeled probe, the reaction mixture is resolved on a non-denaturing polyacrylamide gel and the gel was dried for autoradiography and densitometric scanning.

TUNEL staining Cell sensitivity to rAAV-EGFP or rAAV/CAG-STAP was assayed using the following procedure as in situ cell death Detect Kit (Roche Molecular Biochemicals). Serial sections of 8 μm thickness were prepared from liver tissues that had been fixed in 4% paraformaldehyde and embedded in paraffin.

RT-PCR analysis for determination of gene expression induced by STAP Total RNA was isolated from frozen tissue using RNAzol B. Messanger RNA expression in each sample was determined by reverse transcription-polymerase chain reaction using GeneAmp RNA PCR Core kit (PerkinElmer Life Science).: TIMP-1: 5′-CCA CAG ATA TCC GGT TCG CCT ACA-3′(forward), 5′-GCA CAC CCC ACA GCC AGC ACT AT-3′ (reverse). The cycling parameters were 5 min at 94° C., following by 35 cycles of 1 min at 94° C., 1 min at 55° C. and 1 min 72° C. After amplification, PCR products were electrophoresed on a 1% agarose gel containing ethidium bromide and visualized with UV light. Other primers USED for this study were: Procollagen α type-1: 5′-TAC TAC CGG GCC GAT GAT GC-3′ (forward), 5′-TCC TTG GGG TTC GGG CTG ATG TA-3′ (reverse), procollagen III: 5′-CCC CTG GTC CCT GCT GTG G-3′(forward), 5′-GAG GCC CGG CTG GAA AGA A-3′ (reverse), TGF-β1: 5′-TAT AGC AAC AAT TCC TGG CG-3′ (forward) and 5′-TGC TGT CAC AGG AGC AGT G-3′ (reverse), WPRE: 5′-GCT AAA GAT TCT TGT ATA AAT CCT GGT TGC TGT CT-3′ (forward), 5′-GCA TCT CGA GGA AGG GAC GTA GCA GAA GAA C-3′ (reverse). While G3PDH was used internal control, G3PDH: 5′-CCC TTC ATT GAC CTC AAC TAC ATG G-3′ (forward), 5′-CAT GGT GGT GAA GAC GCC AG-3′ (reverse). c-myc: 5′-CAA ACT GGT CTC CGA GGA GC-3′ (forward), 5′-ACA TGG CAC CTC TTG AGG AC-3′ (reverse); GST-al: 5′-TCT GAA AAC TCG GGA TGA CC-3′ (Forward); 5′-CTG CGG ATT CCC TAC ACA TT-3′ (reverse); GST-α2: 5′-AGA TTG ACG GGA TGA AGC TG-3′(reverse), 5′-GTG CAG CTC CGC TAA AAC TT-3′ (reverse).

In situ hybridization In situ hybridization was carried out as described previously. Dehydrated sections were hybridized overnight at 55° C. with probe solution according to an established in situ hybridization protocol (Ambion). The sections were developed with 1×BCIP/NBT solution (Zymed) to desired intensity. The negative controls were hybridized with Dig-labeled sense cRNA.

Immunohistochemical staining and analysis The liver was soaked in 30% sucrose in PBS and sections 10 μm in thickness were cut on a cryostat and thaw-mounted onto slides. Sections were rinsed three times with PBS containing 0.2% Triton-X100 prior to incubation in 1% H₂O₂ in methanol for 1 min, rinsed three times in PBS, and then incubated with 4% horse serum in PBS for 1 hour. After further PBS-Tween 20 rinses, sections were incubated with the primary antibody overnight at room temperature. Sections were washed with PBS-Tween prior to a 2-hour incubation with secondary antibody for 5 min. The sections were then rinsed with PBS or distilled water before being mounted with Vectashield (Vector La, Calif.). Immunofluorescent signals were captured using a Leica 4d TCS confocal microscope, and images were processed using Adobe Photoshop 5.0. Synthesis of TGF-β1, α-smooth muscle actin and procollagen type I, was examined by immunohistochemistry.

Biochemical analysis Serum albumin, bilirun, aspartate transaminase (AST) (EC2.6.1.) and alanine transaminase (ALT) (EC 2.6.1.2) activities in rat blood were determined in Queen Marry Hospital, Hong Kong.

Masson's trichrome and HE staining Paraffin-embedded sections were stained with Masson's trichrome and hematoxylin-eosin. Liver cirrhosis was determined using computer image analysis techniques on Masson's trichrome-stained histological slides, focusing on the extent of pathological changes including proportions of collapsed hepatocytes, regenerative nodules, distribution of fibrous septa, spread of reticulin fibers, the formation of thin fibrotic septa and a micro-nodular pattern of the parenchyma among the experimental groups. The differences among the area of fibrotic tissue between the treated and the untreated were analyzed.

Statistical analysis Data were given as means±SM. ANOVA were performed to test the significance. P values were considered to be statistically significant when less than 0.05.

Determination of hydroxproline content Hydrolysis was carried out by concentrated hydrochloric acid. Level of hydroxyproline was measured by reversed phase HPLC with fluorometric detection after acid hydrolysis.

Experimental model of common bile duct ligation Male Sprague-Dawley rats (200±20 g) were injected with 5×10¹¹ rAAV-EGFP or rAAV/rSTAP/animal respectively (n=6). At day 3, the common bile ducts were double ligated and scission in between under anesthesia. Sham-operated rats were treated with the same procedure except that the bile was not ligated and scissed (n=6). Following standard protocols, blood samples were taken to determine AST and bilirubin. Rats were sacrificed after rats were subjected to ligation for 28 days. HSC and non-HSC cells were isolated from SD rats following standard procedure as described above for further analysis. Pieces of the liver were fixed in 4% formalin for histological examination. For treatment study, the common bile ducts of male SD rats were double ligated for 12 days prior to injection with 5×10¹¹ rAAV-EGFP or rAAV/rSTAP/animal respectively (n=5). Animals were sacrificed 12 day after injection.

B. Results

Overexpression of STAP Inhibits the in vitro Activation of HSC

The activation of HSC has long been framed as the central event in liver cirrhosis. Preventing HSC activation can slow down and even reverse cirrhosis. Successful targeting to HSC is a key step for an antifibrotic therapy. To ascertain whether HSC can be efficiently transduced with rAAV, freshly isolated primary rat hepatic HSC cells (3 day cultures) were transduced with rAAV containing the 570 bp rat or human STAP (rAAV/rSTAP, rSTAP), or (rAAV/hSTAP, hSTAP). Immuno-histochemical stainning confirmed the trasduction of ˜90% of the primary HSC in culture (desmine staining indicating that 98% of the cells were HSC—). Western blotting confirmed the over expression of STAP in the transduced cells. Chronic oxidative stress and damage is associated with the subsequent induction of liver fibrosis and cirrhosis. Next, it was tested whether the ectopically expressed STAP could function as an effective anti-oxidant during the activation of HSC, thus ameliorating liver cirrhosis. The primary HSC were transduced with rAAV/rSTAP, rAAV/hSTAP or the control rAAV/eGFP vector (MOI: 5×10⁴) and 48 hours later subjected to oxidative stress by an 18 hour exposure to 50 μM Fe-NTA and 20 μM arachidonic acid (Fe/AA treatment). The Fe/AA treatment caused a significant increase (P<0.01, ANOVA) in the levels of both malonaldehyde (MDA, 47.0±15.4 to 94.4±34.0 nmol/grame protein) and 4-hydroxynonenal (4-HNE, 21.8±5.3 to 34.7±5.3 nmol/gram protein, P<0.01, ANOVA) in the untransduced HSC, or those infected with the control vector (rAAV/eGFP). In contrast, there was no statistically significant alterations in the levels of MDA and 4-HNE in the medium of the STAP transduced cells. Indeed, even in the absence of Fe/AA treatment, the STAP transduced cells produced significantly lower levels of MDA and 4-HNE than the control untransduced HSC.

One of most striking features of the Fe/AA treated primary HSC was their increased synthesis of TIMP-1 and TGF-β1 (a pro-fibrogenic factor produced in the activated HSC). The levels of TIMP-1 and TGF-β1 mRNA were determined by RT-PCR. High mRNA levels appeared in the HSC exposed to Fe/AA, while both TIMP-1 and TGF-β1 mRNA levels were suppressed in the STAP transduced HSC. In the absence of Fe/AA treatment, the latter had even lower levels of these factors than the control primary HSC cultured for 14 days. These studies demonstrated that STAP overexpression was able to act as an effective anti-oxidant during the in vitro Fe/AA treatment of HSC.

STAP Induced Changes in AP-1 Binding Activity in vitro

To explore the potential association of AP-1 with induction of oxidative stress and the potential function of STAP following the rAAV/STAP transduction, electrophoretic gel mobility shift assays were used to compare the extent of binding of nuclear proteins isolated from transduced, untransduced and control HSC to an AP-1 consensus sequence. AP-1 binding activity increased markedly in the primary HSC cells after the Fe/AA treatment. Moreover, binding activity in the HSC transduced with rAAV/rSTAP for two days prior to exposure to Fe/AA was even lower than normal. To establish whether this difference in binding activity might also be related to changes in c-jun protein levels, nuclear extracts of normal HSC and HSC transduced with or without rAAV/rSTAP for two days prior to exposure to Fe/AA by western blotting were examined. Immunoblotting with anti-jun revealed higher c-jun levels in the Fe/AA treated HSC while the STAP transduced cells had much lower c-jun levels.

STAP Prevents Oxidation Stress Induced Increases in Nuclear NF-kB

Activation of NF-kB binding is highly responsive to stress stimuli as demonstrated by Super-shift analysis of nuclear extracts prepared from the control HSC or HSC transduced with STAP for two days prior to exposure to the Fe/AA treatment (50 μM Fe-NTA and 20 μM arachidonic acid for 18 hours). Increased levels of nuclear NF-kB, a redox-sensitive transcription factor, were detectable in the Fe/AA treated HSC. This increase was suppressed by the over expression of STAP prior to the induction of oxidative stress. Furthermore, super-shift analysis with antibodies specific to the P65 subunits of NF-kB revealed that the mobility of these binding complexes were further retarded, indicating that p65/p50 heterodimers and possibly p65 homodimers accumulated in the nucleus following the Fe/AA treatment, suggesting that oxidation stress mediated increases in the nuclear levels of proteins that bind the NF-kB response elements can be blocked by the over expression of STAP in HSC.

The in vivo Transduction of Liver with rAAV/STAP

To assess the in vivo effect of increased expression of STAP in the liver, 3×10¹¹ vector particles were delivered into the portal vein of each male SD rat. The animals were sacrificed 4 weeks later and in situ hybridization studies detected STAP transcripts, but mainly in the periportal regions of the liver samples obtained from the rAAV/STAP infected animals. Immuno-histochemical staining of the hepatic tissue confrimed the increased expression of STAP protein in the rAAV/rSTAP infected rat livers. As with the STAP transcription, protein expression was restricted mainly to the periportal areas of the transduced livers. Double staining of the tissue sections with desmin strengthened the suggestion of the preferential transduction of HSC, rather than hepatocytes, by the rAAV-2.

The strongest STAP immuno-reactivity was found in the liver sections of chronic CCl₄ treated animals where both HSC and injured hepatocytes were stained positive. Although STAP shares about 40% amino acid sequence homology with the haemoglobulin and myoglobin family of proteins, the antibody used recognizes the N-terminal 21 amino acids of STAP which has no homology with the haemoglobin/myogloin family members. Western immunobloting analysis of STAP in the normal animals exposed to chronic CCl₄ induced injury demonstrated the high level presence of STAP as a dimer. In contrast, in the rAAV/STAP livers, STAP was primarily in a monoimeric form. The presence of this monomeric form of STAP and/or its continuous and elevanted expression by prior transduction of the HSC, may be responsible for its ability to protect against CCl₄ induced liver cirhosis.

STAP Gene Expression Prevents CCl₄ Induced Liver Cirrhosis

To examine the potential of STAP to suppress damage induced liver fibrosis in vivo, thus preventing exacerbated fibrosis in animals, the rAAV/rSTAP, rAAV/hSTAP, rAAV/eGFP or the equivalent volume of the carrier PBS were delivered to male SD rats as described (n=10 for each group).

Histological examination demonstrated a similar architecture in the hepatic tissue of the CCl₄-rAAV/rSTAP & CCl₄-rAAV/hSTAP rats and the normal untreated animals (FIG. 3a). In contrast, the liver architectures were distorted in both CCl₄-control & CCl₄-rAAV/eGFP rats. The distortion was marked by extensive fibrotic replacement (FIG. 3b, c) with a micronodular pattern throughout the liver parenchyma. The parenchymal cells also had a “collapsed” appearance and regenerative nodules, separated by fibrous septa and radial reticulin fibers, were present. The formation of thin fibrotic septa joining the central areas was observed, and a micronodular pattern was evident in the liver parenchyma. Computer-aided imaging was used to determine the fibrosis index by quantifying the proportion of collagen-I positive areas. Although the fibrosis index in the CCl₄-rAAV/rSTAP and CCl₄-rAAV/hSTAP rats were about two fold higher than the index in the control animals, these values were less than half the index for the CCl₄-rAAV/eGFP and less than one third the values for the uninfected animals that were treated with CCl₄ (FIG. 3e). Fibrous connective tissue components in Glisson's sheath and the pseudo-lobular formations found in the cirrhosis of untreated animals were inhibited by STAP vector transduction. Furthermore, RT-PCR analysis of hepatic tissue isolated from these animals revealed dramatically increased procollagen-I levels in the CCl₄-control & CCl₄-AAV/eGFP rats. This was in contrast to the similarly low levels of procollagen-I in the CCl₄-rAAV/rSTAP, CCl₄-rAAV/hSTAP and normal rats. Consistent with the histological evidence of fibrosis, increased levels of TGF-β1 (a major pro-fibrogenic factor produced in activated HSC) transcripts and protein were present in the CCl₄-control and CCl₄-AAV/eGFP rats. That the activated HSC were contributing to the enhanced TGF-β1 production was further corroborated by the predominantly centrilobular TGF-β1 staining, corresponding to the positive staining of these cells with α-smooth muscle actin and desmin, both markers of activated HSC. The CCl₄-induced increase in TGF-β1 mRNA and protein expression were reduced markedly by rAAV/STAP transduction in the CCl₄-rAAV/rSTAP & CCl₄-rAAV/hSTAP rats. In addition, increased numbers of desmin positive HSC were detectable in the fibrotic regions of the liver in the CCl₄-control and CCl₄-AAV/eGFP, many of which were transformed into α-SMA positive myofibroblast-like cells. Further support for oxidative stress induced TGF-β1 expression in HSC was the up-regulation of TGF-β1 and the increased expression for both procollagen-I and SMA in these cells.

It was next determined whether transgenic STAP expression could prevent the TGF-β1 induced apoptosis in hepatocytes, another well-documented downstream pathogenetic feature of oxidative stress. TUNEL staining revealed the presence of apoptotic cells in the liver sections of all experimental groups. However, STAP gene expression clearly prevented CCl₄ induced hepatocyte apoptosis as the numbers of apoptotic cells in the liver sections of CCl₄-rAAV/rSTAP & CCl₄-rAAV/hSTAP rats were similar to the levels in normal livers, while the CCl₄ treated and AAV/eGFP animals had a 2 folds higher level of apoptosis. Therefore, the transgenic expression of STAP by portal vein delivery of rAAV/STAP was able to prevent CCl₄ induced liver cirrhosis.

In order to further substantiate the protective effect of transgenic STAP expression, serum aspartate aminotransferase (AST) and alanine amino-transferase (ALT) levels were determined. Similar levels of both AST and ALT were present in the serum of the normal animals and the CCl₄ treated rAAV/rSTAP and rAAV/hSTAP animals, suggesting normal liver function in the animals treated with rAAV/STAP and the absence of significant liver necrosis damage that was induced by the CCl₄ treatment of the normal animals. Inhibition of HSC activation by STAP was further supported by the presence of near normal levels of α-SMA protein and TIMP-1 mRNA in the CCl₄ treated rAAV/STAP animals.

To clarify whether modulation of AP-1 DNA binding activity in vivo is involved the STAP conferred protection, we analyzed the nuclear extracts of liver tissues isolated from these experimental groups of animals. Transgenic expression of STAP clearly correlated with decreased binding of nuclear proteins to the AP-1 consensus sequence oligo-nucleotide probe. Similarly, the induction of c-myc mRNA increase, as determined by RT-PCR, was inhibited by the transgenic STAP expression in treatment groups. These changes were not likely to be non-specific as highlighted by the absence of GST-α1 mRNA alterations in any of the experimental groups but their decrease in the GST-α2 mRNA levels in the CCl₄-control and CCl₄-AAV/eGFP rats, but not in the CCl₄-rAAV/rSTAP and CCl₄-rAAV/hSTAP rats. Since GSTs constitute the endogenous peroxidase activities in quiescent HSC, this data demonstrates the ability of the transgenic STAP to act as an effective anti-fibrotic scavenger of peroxides, able to inhibit the activation of HSC.

STAP Overexpression Ameliorates Progressive Liver Damage Initiated by Previous Exposure to CCl₄

Accumulative data from clinical and laboratory based research data support that early stages of cirrhosis could be reversible. To explore whether ectopic STAP expression could reverse evolving liver cirrhosis in our paradigm, CCl₄-rats were injected intraportally with 3×10¹¹ rAAV/rSTAP, rAAV/hSTAP or rAAV/eGFP (n=10) respectively after completing the 8-week course of CCl₄ injections. These animals were then sacrificed for analysis after another four weeks, during which CCl₄ was given continuously. Histology and immunochemistry examinations revealed a similar trend in the experimetnal rats as found in the prevention study. Despite consecutive induction of CCl₄, STAP administration led to a clear healing process that involved the clearance of necrotic/apoptic cell debris and remodeling of the extracellular matrix when compared with those of CCl₄-control or rAAV/EGFP at week 8 and at week 12. In contrast, those of rAAV/eGFP rats revealed progressive changes in the hepatic histology, with futher increases in both of TGF-β1 and α-SMA positive cells which were widely distributed and formed several radial networks. Since overproduction of TGF-β1 is a chief cause of tissue fibrosis in various organs. Collapse of parenchymal cells and the formation of regenerative nodules continued, and thickening of reticulin fibers were also evident. However, these features were remarkably reverted by the ectopic STAP expression in rAAV/rSTAP and rAAV/hSTAP rats with minimal residual fibrosis in the periportal and centrilobular liver and absence of obvious deformation of the liver architecture.

The rAAV Driven STAP gene therapy also resulted in improvement in hepatic function.

Biochemical analysis revealed the serum ALT levels in the CCl₄ treated rAAV/eGFP animals increased continuely from 1,603±397 U/L at week 8 to 2,080±110 U/L at week 12, and was about 30 fold higher than normal. These were dramatically reduced to 67±15 U/L for rAAV/hSTAP rats and 99±18 U/L for rAAV/rSTAP rats, and were nearly normal or in the normal range. Similarly, serum AST levels in rAAV/EGFP increased continuously, and was about 17 fold higher than those of normals, while levels of AST in both rAAV/rSTAP and rAAV/hSTAP rats decreased from 1,280±265 U/L (CCl₄-control) at week 8 to 179±37 U/L for rAAV/rSTAP and 198±25 U/L for rAAV/hSTAP at week 12, and was about 2 folds higher than normal. These important changes were accompanied by reduction of fibrosis and a return to normal liver architecture in both rAAV/rSTAP and rAAV/hSTAP. Such a change was not observed in rAAV/eGFP group. Therefore, the data show promise that liver fibrosis can be ameliorated by STAP administration.

STAP Overexpression Ameliorates Progressive Liver Damage Initiated by Common Bile Duct Ligation

To explore whether ectopic STAP expression could attenuate evolving liver fibrosis in other animal model. Male Sprague-Dawley rats (200±20 g) were injected with 5×10¹¹ rAAV-EGFP (BDL-eGFP) or rAAV/rSTAP (BDL-STAP)/animal respectively (n=7). At day 3, the common bile ducts were double ligated and scission in-between under anesthesia. Sham-operated rats were treated with the same procedure except that the bile was not ligated (n=7). Twenty eight days after BDL, rats pretreated with rAAV/Egfp (n=7) had significant cholestatic liver injury demonstrated by histological evidence of extensive fibrosis with nodule development (FIG. 12b). All rats in this group progressively developed ascites and two died, on days 21 and 27. However, all rats receiving rAAV/rSTAP prior to BDL (n=7) remained alive and free of ascites, although liver histology showed bile duct proliferation and concentric periductal fibrosis, the liver architecture was substantially preserved (FIG. 12D). Overexpression of STAP in HSC, prior to the induction of cholestatic liver injury, reduced the degree of liver dysfunction, as assessed by total bilirubin (sham: 2.1±0.8 μmol/L; rAAV/rSTAP: 51.6±30.1 μmol; and rAAV/eGFP: 99.9±24.2 μmol) and AST (sham: 74.3±28.9 U/L; rAAV/rSTAP: 407±209 U/L; rAAV/eGFP: 807±357 U/L). Hydroxyproline content was 0.09±0.03 mg/g for shamed, 0.42±0.26 mg/g for BDL-STAP and 0.87±0.43 mg/g liver tissue for BDL-eGFP.

A similar therapeutic effect was observed when BDL was carried out 12 days prior to pv injection of rAAV/eGFP (n=5) or rAAV/rSTAP (n=5). Liver histology, at the time of sacrifice a further 12 days later, showed markedly less fibrosis in those receiving rAAV/rSTAP (FIG. 14D) compared to rAAV/eGFP (FIG. 13B). STAP gene therapy after the onset of cholestatic liver injury, reduced the degree of liver dysfunction, as assessed by total bilirubin (sham 2.9±1.0 μmol/L compared to rAAV/rSTAP 77.3±35.0 μmol, P<0.05; and rAAV/eGFP 130±11.3 μmol, P<0.05) and AST (sham 74.3±28.9 U/L compared to rAAV/rSTAP 497±253 U/L, P=0.0668; and rAAV/eGFP 1,113±112 U/L, P<0.01 compared with sham and P=0.065 compared with rAAV/STAP), and induced a quiescent phenotype in HSC, isolated from liver at the time of sacrifice, as assessed by real time RT-PCR analysis of levels of TGF-β1 and PC-1 transcripts (FIG. 14A-14D).

Long-Term Effect and Safety of STAP Expression

To estimate potential of STAP application in human liver fibrosis therapy, we established a new set of experiment to monitor the long-term effect and safety. CCl₄-rats were injected intraportally with 3×10¹¹ rAAV/rSTAP and rAAV/eGFP respectively (n=5) after completing the 8-week course of CCl₄ injections. Animals were subjected to another four weeks consective CCl₄ induction, and then were kept under the normal condition for 40 weeks prior to sacrifice. A group of normal served as control. We found that as previous report for CCl₄ induced animals all examined animals appear normal in gross appearance and behaviror. All animals survived well except that CCl4-eGFP in which two rats were death during the experimental period. No tumour or abnormal appearance was found in CCl4-rSTAP group. There were not significant differences in body weight among three experimental groups, but a substantial accumulation of fat in abdominal cavity of both CCl₄-eGFP and CCl₄-STAP groups but not in normal group. Previous investigators noted that side effect of CCl₄ induction resulted in an increase in fat accumulation in induced animals. To determine actual effect of induction of CCl₄ and STAP expression on liver structure, Sections of liver tissues from different group were subject histology and immuno-staining analysis, administration of rAAV/STAP significantly attenuated liver damage and fibrosis. There were still signs of fibrosis in rAAV/STAP group, but accumulative collagen network can not be found in all sections of rAAV-STAP group (FIG. 15E-15F). Furthermore, histological sections of livers revealed that all rAAV-STAP had been healing, although complete resolution of fibrosis at the end is not clear. In contrast, accumulative collagen network still can be found in the all sections of CCl₄-eGFP had a characteristic appearance, i.e. were enlarged, hard and nodular due to widespread hepatic fibrosis after discontinuation of treatment with CCl₄ for 40 weeks (FIG. 15A-15B). Hydroxyproline content for normal group was 0.268±0.05 mg/g liver tissue for normal, 0.309±0.051 mg/g liver tissue for rAAV/STAP group and 0.387±0.06 mg/g liver tissue for CCl₄-eGFP. Taking all data together, TSAP is a very promising agent for liver fibrosis therapy.

C. Discussion

Transformed (activated) sinusoidal HSC are the prime source of pathologic deposits of extracellular matrix in hepatic fibrosis triggered by insults ranging from viral infections, metabolic stress, biliary obstruction and hereditary defects. Experimental and clinical data have suggested that hepatic fibrosis and early cirrhosis may be reversible, thereby encouraging the development of therapeutic strategies targeting specifically at HSC. Attempts have been made to block the activation of quiescent HSC, to induce apoptosis of activated HSC or myofibroblasts, and to deliver agents to activated HSC by coupling them to cyclic peptide binding to cell surface collagen VI receptors upregulated in these cells.

To confirm the therapeutic role of candidate intracellular molecular pathways responsible for the initiation and maintenance of progressive hepatic fibrosis, the capability to selectively target different major cell types in vivo is crucial. rAAV-2 has been shown to transduce HSC with high efficiency in vitro, making it an attractive vector for HSC targeting. Previous study has shown that transduction efficiency of HSC insolated from the normal liver for adenovirus was <60%. The transduction of primary hapetic cells with an identical construct and MOI of rAAV-1, rAAV-2 and rAAV-8 containing a reporter gene, eGFP, revealed that rAAV-2 was the most efficient agent for transduction of HSC. All these data with recent report that only up to 5% transduction efficiency of hepatocytes with rAAV-2 in vivo and the preferential transduction of the periportal tissue by rAAV-2, suggest that rAAV-2 could effectively target HSC in vivo.

No definitive cure for liver cirrhosis has yet been developed. The possibility of using uPA, HGF and telomerase to treat cirrhosis in human patients has been studies, but doubts have been expressed whether this approach can be applied safely. Targeting HSC offers a tempting alternative approach. With efficient selective transduction of HSC established in vivo, we are enable to examine the effect of an important liver specific anti-oxidant molecule, STAP.

STAP was originally isolated by comparative proteomic study of thioacetamide-induced fibrotic liver. Similar induction of STAP carbon tetrachloride induced hepatic fibrosis was found by both immunoblotting and immunocytochemical analyses. In sharp contrast to the rAAV driven increase in STAP expression in HSC, the endogenous STAP upregulation failed to confer the anti-fibrosis protection. One possible explanation was that the predominant endogenous dimer form of STAP induced by CCl₄ was either not active or much less potent as the monomeric STAP biologically, suggested by the predominant monomeric form in rAAV/STAP driven over-expression in vivo and the peroxidase activity of monomeric hSTAP and its ability to suppress conjugated diene formation in a dose-dependent manner.

The liver is highly metabolic and is responsible for metabolising drugs/xenobiotics, thus putting itself at increased risks from oxidative stress as a result of the formation of ROS. One of the mechanisms recently described links oxidative stress to nuclear signaling in HSC and hence the pathogenesis of hepatic fibrosis. In line with over-expression of STAP stabilizing the levels of HNE and MDA in HSC exposed to ROS, we propose that STAP could protect against cellular damage caused by HNE, MDA, or other ROS by scavenging damaging free radicals.

The GSTs is a key component of the endogenous anti-oxidative system in HSC that rapidly convert products of lipid peroxidation such as HNE to glutathione conjugates, a basal function critical to the highly metabolic liver. The activation or transformation of HSC could contribute to amplifying the impact of additional stress from the primary insults. It was reasoned that the normalization of GSTa2 in STAP over-expressed CCl₄-induced chronic model indicated that CCl₄-induced chronic animals lack major forms of GST and therefore had a limited ability to detoxify ROS. Thus compounds like HNE and MDA could accumulate and thereby affect additional critical cellular functions, resulting in increased extracellular matrix deposition. Transduction of STAP into CCl₄-induced chronic animals allowed GST mRNA level of HSC to normalize. Loss of GSTs could be prevented, the activated HSC would be more resistant to oxidant stress, and therefore the index of collagen I positive areas in the animals treated either with rAAV/STAP or rAAV/hSTAP vectors was very close to that of normal rats. Moreover, the activation of HSCs may be associated with long-term and sustained modulation of transcriptional and/or post-transcriptional events involved in the regulation of GSTs mRNA expression. STAP could thus play a role as an antifibrotic scavenger of peroxides in the liver.

NF-kB, like other transcription factors, is sensitive to oxidative modification of its cysteine residue at position 62 in the p50 subunit, which is crucial for DNA-binding activity. Oxidation of these crucial cysteine residues frequently results in the inhibition of transcription factor activity by oxidative stress. It has been revealed that NF-kB-binding sites are in the promoter region of GM-CSF, TNF-β1, IL-6 and growth factors relevant to inflammation, whereas the gene activation of TGF-β1, the most fibrogenic cytokines together with PDGF, occurs through binding to the AP-1 site present on its long terminal repeat. Activation of NF-kB binding in exposure to MDA and HNE indicate stress signaling pathway is involved in redox-sensitive factor NF-kB. Over expression of STAP in HSC transduced with STAP vectors leads to a decrease in NF-kB binding, suggesting that STAP suppresses activation of HSC via NF-KB pathway. Scavenging of radical-derived organic peroxides by STAP could be an adaptive reaction to normalize the cellular redox status during the cell activation.

In summary, it was demonstrated that transduction of STAP reduced or suppressed levels of TGF-β1 and α-SMA, leading to the prevention of rat liver cirrhosis-induced CCl₄. Stable gene transduction from one dose of rAAV could prevent liver cirrhosis (FIG. 5c). Protection against cellular damage was achieved by overexpression of STAP mainly in HSC via AP-1, NF-kB, c-myc and probably other multiple mechanisms of the scavenging of radical-derived organic peroxides during liver cirrhosis. Pattern of changes in histology, immunochemistry and biochemistry revealed a similar trend in the experimental rats as found in the prevention study. This study establishes a novel approach to target HSC using rAAV vector containing a hepatic anti-oxidation gene and offers potentials for the development of a gene therapy for patients with progressive liver cirrhosis.

### REFERENCES

1. Aguilar-Delfin, I., Lopez-Barrera, F., and Hernandez-Munoz R. (1996) Selective enhancement of lipid peroxidation in plasma membrane in two experimental models of liver regeneration: partial hepatectomy and acute CCl₄ administration. Hepatology 24(3):657-662.
2. Asahina K., Kawada N., Kristensen D. B., Nakatani K., et al. (2002) Characterization of human stellate cell activation-associated protein and its expression in human liver. Biochim. Biophys. Acta. 1577(33):471-475.
3. Bahr, M. J., et al. (1999) Control of the tissue inhibitor of metalloproteinases-1 promoter in culture-activated rat hepatic stellate cells: regulation by activator protein-1 DNA binding protein. Hepatology 29:839-848.
4. Beljaar, L. et al., Successful targeting to rat hepatic stellate cells using albumin modified with cyclic peptides that recognize the collagen type VI receptor J. Biol. Chem 275,12743-12751 (2000).
5. Di Sario, A. et al. Characterization of ion transport mechanisms regulating intracellular PH in hepatic stellate cells. AM J Physiol. 273, G39-48 (1997).
6. During, M. J., Xu, R. A., Young, D., Kaplitt, M. G., et al. (1998) Peroral gene therapy of lactose intolerance using an adeno-associated virus vector. Nat. Med. 4:1131-1136.
7. Friedman, S. L. (1993) The cellular basis of hepatic fibrosis. Mechanisms and treatment strategies. N. Engl. J. Med. 328(25):1828-35.
8. Friedman, S. L. et al., Isolated hepatic lipocytes and Kupffer cells from normal human liver: morphological and functional characteristics in primary culture. Hepatology 15,234-43 (1992).
9. Friedman, S. L., The cellular basis of hepatic fibrosis: Mechanisms and treatment strategies N Engl J. Med. 328 (25), 1828-35 (1993).
10. Friedman, S. L., Liver fibrosis-from bench to bedside J Hepatology 38, S38-S53 (2003).
11. Gao, G. P. et al., Novel adeno-associated virus from rhesus monkeys as vectors for human gene therapy, PNAS, 99,11854-11859 (2002).
12. Gao, G. P., et al. (2002) Novel adeno-associated virus from rhesus monkeys as vectors for human gene therapy. Proc. Natl. Acad. Sci. USA 99:11854-11859 (2002).
13. Iredale, J. P. New research provides a basis for rational and targeted treatment BMJ 327, 143-7 (2003).
14. Kawada, N., Krisensen, D. B., Asahina, K., Nakatani, K., et al. (2001) Characterization of a stellate cell activation-associated protein (STAP) with peroxidase activity found in rat hepatic stellate cells. J. Biol. Chem. 276:25318-25323.
15. Kim, S J et al., Activation of the second promoterof the transforming growth factor-β1 and phorbol ester occurs through the same target sequences, J. Biol. Chem. 264,19373-19378, (1989).
16. Lee K. S. et al. (1995) Activation of hepatic stellate cells by TGFα and collagen type I is mediated by oxidative stress through c-myb expression, J. Cli. Inv. 96:2461-2468.
17. Lee, M. Y., Hepatitis B virus infection N. Engl. J. Med, 24, 1733-1745 (1997).
18. Libermann, T. A. & Baltimore, D., Activation of the interleukin-6 gene expression through the NF-kappa B transcription factor, Mol cell Biol, 10, 2327-2334, (1990).
19. Mari M. and Cederbaum, A. I. (2000) Cyp2E1 overexpression in HepG2 cells induces glutathione synthesis by transcriptional activation of γ-glutamylcysteine synthetase. J. Biol. Chem. 275:15563-15571.
20. Mendelson K. G. et al. (1996) Independent regulation of JNK/p38 intogen-activated protein kinases by metabolic oxidative stress in the liver. Proc. Natl. Acad. Sci. USA 93:12908-12913.
21. Messer, G., Weiss, E. H. & Baeuerle, P. A. Tumor necrosis factor beta (TNF-beta) induces binding of the NF-kappa B element in the TNF-beta promoter, Cytokine, 2, 1-9, (1990).
22. Miao, C. H. et al., Nonrandom transduction of recombinant adeno-associated virus vectors in mouse hepatocytes in vivo: cell cycling does not influence hepatocyte transduction, J. Virol. 74, 3793-3803 (2000).
23. Mitomo, K., Nakayama, K, Fujimoto, K, Sun .X, Seki, S & Yamamoto, K. Two different cellular redox systems regulate the DNA-binding activity of the p50 subunit of NF-kappa B in vitro, Gene, 145,197-203, (1994).
24. Nieto et al. (2001) Rat hepatic stellate cells contribute to the acute-phase response with increased expression of alpha1 (I) and alpha1 (IV) collagens, tissue inhibitor of metalloproteinase-1, and matrix-metalloproteinase-2 messenger RNAs. Hepatology 33(3):597-607.
25. Parola et al. (1998) HNE interacts directly with JNK isoforms in human hepatic stellate cells. J. Clin. Invest. 102:1942-1950.
26. Poli, G. and Parola, M. (1997) Oxidative damage and fibrogenesis. Free Radical Biol. Med. 22:287-305.
27. Rudolph, K. L., et al., (2000) Inhibition of experimental liver b cirrhosis in mice by telomerase gene therapy. Science 287:1253-1258.
28. Salgado, et al. (2000) Liver cirrhosis is reversed by urokinase-type plasminogen activator gene therapy. Mol. Ther. 2:545-551.
29. Salgado, S. et al., Liver cirrhosis is reversed by urokinase-type plasminogen activator gene therapy, Mol. Ther. 2, 545-551 (2000).
30. Schoonbroodt, S. & Piette, J., Oxidative stress interference with the nuclear factor-kB activation pathways, Biochem. Pharmacol. 60,1075-1083 (2000).
31. Schreck, R. & Baeuerle, P. A., NF-kappa B as inducible transcriptional activator of the granulocyte-macrophage colony-stimulating factor gene Mol Cell Biol 10,1281-1286 (1990).
32. Svegliati-Baroni, G., et al. (1999) Hepatology 29:1743-1751.
33. Toledano, MB & Leonard. W J, Modulation of transcription factor NF-kappa B binding activity by oxidation-reduction in vitro PNAS, 88, 4328-4332 (1991)
34. Uchida, K., Shiraishi, M., Naito, Y., Torii, Y., Nakamura, Y & Osawa, T., Activation of stress signaling pathways by the end product of lipid perroxidation, J. Biol. Chem. 274, 2234-2242, (1999).
35. Ueki, T. et al., Hepatocyte growth factor gene therapy of liver cirrhosis in rats Nature Med., 5, 226-230 (1999).
36. Whalen R. et al. (1999) Activation of rat hepatic stellate cells leads to loss of glutathione S-transferase and their enzymatic activity against products of oxidative stress. Hepatology. 30:927-933.
37. Xu, L., Daly, T., Gao, C., Flotte, T. R., et al. (2001) CMV-β-actin promoter directs higher expression from an adeno-associated viral vector in the liver than the cytomegalovirus or elongation factor 1α promoter and results in therapeutic levels of human factor X in mice. Human Gene Ther. 12:563-573.
38. Xu, R. A. et al., Long-term expression of angiostatin suppresses liver matastatic cancer in mice. Hepatology 37,1451-1460 (2003)
39. Xu, R. A. et al., Quantitative comparison of expression with adeno-associated virus (AAV-2) brain-specific gene cassettes, Gene Therapy, 8,1323-1332 (2001).
40. Xu, R. A., Cai, K. X., Zheng, D. X., Ma, H. & Fan, S. T. Molecular therapeutics of HBV, Cur. Gene Ther. 3, 341-356 (2003)
41. Yu, Q., Gue, L. G. & Rockey, D. C. Adenovirus-mediated gene transfer to nonparenchymal in normal and injured liver, Am J. Physiol Gastrointest Liver Physiol 282(3) G565-72 (2002).
42. Zhao, T. et al., The role of human glutathione S-Ttransferases hGSTA1-1 and hGSTA2-2 in protection against oxidative stress, Arch Biochem Biophy, 367, 216-224 (1999).

1. A method for treating liver cirrhosis in a subject comprising administering to the subject a therapeutically effective amount of a rAAV/CAG-STAP vector to treat liver cirrhosis in the subject.

2. The method of claim 1, wherein the rAAV/CAG-STAP vector transduces hepatic stellate cells.

3. The method of claim 2, wherein the transduction of hepatic stellate cells results in the suppression of α-SMA, collagen, and/or TGF-β expression.

4. The method of claim 1, wherein the rAAV/CAG-STAP vector comprises the rat STAP sequence.

5. The method of claim 4, wherein the rAAV/CAG-STAP vector comprises rAAV/CAG-rat STAP vector (CCTCC Patent Deposit Designation V200306).

6. The method of claim 1, wherein the rAAV/CAG-STAP vector comprises the human STAP sequence.

7. The method of claim 1, wherein the rAAV/CAG-STAP vector comprises rAAV/CAG-human STAP vector (CCTCC Patent Deposit Designation V200305).

8. The method of claim 7, wherein the subject is a human.

9. The method of claim 1, wherein the subject is a mammal.

10. The method of claim 9, wherein the mammal is a human.

11. The method of claim 1, wherein the transduction of hepatic stellate cells inhibits fibrogenesis, hepatocyte apoptosis, or both.

12. The method of claim 2, wherein transduction of hepatocytes with STAP reduces ALT and AST levels.

13. A method for preventing or retarding the development of liver cirrhosis in a subject at risk for liver cirrhosis comprising administering to the subject a prophylactically effective amount of a rAAV/CAG-STAP vector to prevent or retard the development of liver cirrhosis in the subject.

14. The method of claim 13, wherein the rAAV/CAG-STAP vector transduces hepatic stellate cells.

15. The method of claim 14, wherein the transduction of hepatic stellate cells results in the suppression of α-SMA, collagen, and/or TGF-β expression.

16. The method of claim 13, wherein the rAAV/CAG-STAP vector comprises the rat STAP sequence.

17. The method of claim 16, wherein the rAAV/CAG-STAP vector comprises rAAV/CAG-rat STAP vector (CCTCC Patent Deposit Designation V200306).

18. The method of claim 13, wherein the rAAV/CAG-STAP vector comprises the human STAP sequence.

19. The method of claim 18, wherein the rAAV/CAG-STAP vector comprises rAAV/CAG-human STAP vector (CCTCC Patent Deposit Designation V200305).

20. The method of claim 12, wherein the subject is a mammal.

21. The method of claim 20, wherein the mammal is human.

22. The method of claim 13, wherein the transduction of hepatic stellate cells inhibits fibrogenesis, hepatocyte apoptosis, or both.

23. The method of claim 14, wherein transduction of hepatocytes with STAP reduces ALT and AST levels.

24. A method for treating liver cirrhosis in a subject afflicted with liver cirrhosis, comprising administering to the subject a therapeutically effective amount of a gene encoding the stellate cell activation-associated protein (STAP), to treat cirrhosis in the subject.

25. A method for preventing or retarding the development of liver cirrhosis in a subject at risk for liver cirrhosis, comprising administering to the subject a prophylactically effective amount of a gene encoding the stellate cell activation-associated protein (STAP), to prevent or retard the development of liver cirrhosis in the subject.

26. A viral vector comprising the rAAV/CAG-rat STAP vector (CCTCC Patent Deposit Designation V200306).

27. A kit comprising the viral vector of claim 25, and instructions for use.

28. A viral vector comprising the rAAV/CAG-human STAP vector (CCTCC Patent Deposit Designation V200305).

29. A kit comprising the viral vector of claim 27, and instructions for use.

30. A pharmaceutical composition comprising the viral vector of claim 26 and a pharmaceutically acceptable carrier.

31. A pharmaceutical composition comprising the viral vector of claim 28 and a pharmaceutically acceptable carrier.

32. A method for treating liver cirrhosis in a subject comprising administering to the subject a therapeutically effective amount of a viral vector including an antioxidant gene, to treat liver cirrhosis in the subject.

33. The method of claim 32, wherein the viral vector transduces hepatic stellate cells.

34. The method of claim 32, wherein the antioxidant gene is catalase.

35. The method of claim 32, wherein the antioxidant gene is SOD.

36. The method of claim 32, wherein the antioxidant gene is STAP.


SEQUENCE LISTING (38 sequences)

SEQ ID NO: 1 - DNA, 20 bp, Artificial Sequence
  Insulin Oligonucleotide Primer In-1
cagcctttgtgaaccaacac

SEQ ID NO: 2 - DNA, 20 bp, Artificial Sequence
  Insulin Oligonucleotide Primer In-2
gcgtctagttgcagtagttc

SEQ ID NO: 3 - DNA, 19 bp, Artificial Sequence
  Beta-actin Primer A-1
ctcttccagccttccttcc

SEQ ID NO: 4 - DNA, 19 bp, Artificial Sequence
  Beta-actin Primer A-2
gtcaccttcaccgttccag

SEQ ID NO: 5 - DNA, 21 bp, Artificial Sequence
  Primer
atggagaaagtgccgggcgac

SEQ ID NO: 6 - DNA, 21 bp, Artificial Sequence
  Primer
tggccctgaagagggcagtgt

SEQ ID NO: 7 - DNA, 24 bp, Artificial Sequence
  Probe
agcatgagtcagacacctcttggc

SEQ ID NO: 8 - DNA, 22 bp, Artificial Sequence
  Probe
agttgaggggactttcccaggc

SEQ ID NO: 9 - DNA, 26 bp, Artificial Sequence
  Probe
ggatccagcgggggcgagcggggcga

SEQ ID NO: 10 - DNA, 20 bp, Artificial Sequence
  Probe
tgcagattgcgcaatctgca

SEQ ID NO: 11 - DNA, 20 bp, Artificial Sequence
  Primer c-Met (forward)
gcaccccaaagctggtaata

SEQ ID NO: 12 - DNA, 20 bp, Artificial Sequence
  Primer c-Met (reverse)
ccggttgaacgatcactttt

SEQ ID NO: 13 - DNA, 20 bp, Artificial Sequence
  Primer HGF (forward)
cgagctatcgcggtaaagac

SEQ ID NO: 14 - DNA, 20 bp, Artificial Sequence
  Primer HGF (reverse)
ggtggttcccctgtaacctt

SEQ ID NO: 15 - DNA, 20 bp, Artificial Sequence
  Primer Procollagen alpha type-1 (forward)
tactaccgggccgatgatgc

SEQ ID NO: 16 - DNA, 23 bp, Artificial Sequence
  Primer Procollagen alpha type-1 (reverse)
tccttggggttcgggctgatgta

SEQ ID NO: 17 - DNA, 19 bp, Artificial Sequence
  Primer Procollagen III (forward)
cccctggtccctgctgtgg

SEQ ID NO: 18 - DNA, 19 bp, Artificial Sequence
  Primer Procollagen III (reverse)
gaggcccggctggaaagaa

SEQ ID NO: 19 - DNA, 25 bp, Artificial Sequence
  Primer MMP-13 (forward)
agcttggccactccctcggtctgtg

SEQ ID NO: 20 - DNA, 24 bp, Artificial Sequence
  Primer MMP-13 (reverse)
gtctcgggatggatgctcgtatgc

SEQ ID NO: 21 - DNA, 20 bp, Artificial Sequence
  Primer TGF-Beta1 (forward)
tatagcaacaattcctggcg

SEQ ID NO: 22 - DNA, 19 bp, Artificial Sequence
  Primer TGF-Beta1 (reverse)
tgctgtcacaggagcagtg

SEQ ID NO: 23 - DNA, 24 bp, Artificial Sequence
  Primer Tl1 (forward)
ccacagatatccggttcgcctaca

SEQ ID NO: 24 - DNA, 22 bp, Artificial Sequence
  Primer Tl1 (reverse)
gcacaccccacagccagcacta

SEQ ID NO: 25 - DNA, 35 bp, Artificial Sequence
  Primer WPRE (forward)
gctaaagattcttgtataaatcctggttgctgtct

SEQ ID NO: 26 - DNA, 31 bp, Artificial Sequence
  Primer  WPRE (reverse)
gcatctcgaggaagggacgtagcagaagaac

SEQ ID NO: 27 - DNA, 20 bp, Artificial Sequence
  Primer Zf9 (forward)
acaaccaggaagacctgtgg

SEQ ID NO: 28 - DNA, 20 bp, Artificial Sequence
  Primer Zf9 (reverse)
tgctttcaagtgggagcttt

SEQ ID NO: 29 - DNA, 25 bp, Artificial Sequence
  Primer G3PDH (forward)
cccttcattgacctcaactacatgg

SEQ ID NO: 30 - DNA, 20 bp, Artificial Sequence
  Primer G3PDH (reverse)
catggtggtgaagacgccag

SEQ ID NO: 31 - DNA, 24 bp, Artificial Sequence
  Primer
ctatggccctgaagagggcagtgt

SEQ ID NO: 32 - DNA, 23 bp, Artificial Sequence
  Primer
gcacaccccacagccagcactat

SEQ ID NO: 33 - DNA, 20 bp, Artificial Sequence
  Primer
caaactggtctccgaggagc

SEQ ID NO: 34 - DNA, 20 bp, Artificial Sequence
  Primer
acatggcacctcttgaggac

SEQ ID NO: 35 - DNA, 20 bp, Artificial Sequence
  Primer
tctgaaaactcgggatgacc

SEQ ID NO: 36 - DNA, 20 bp, Artificial Sequence
  Primer
ctgcggattccctacacatt

SEQ ID NO: 37 - DNA, 20 bp, Artificial Sequence
  Primer
agattgacgggatgaagctg

SEQ ID NO: 38 - DNA, 20 bp, Artificial Sequence
  Primer
gtgcagctccgctaaaactt